(19) United States
(12) Patent Application Publication
(10) Pub. No.: US 2025/0352609 A1
(43) Pub. Date: Nov. 20, 2025

(54) Methods For Treating Alzheimer's Disease or Dementia Using A Calcilytic And A Calcimimetic

(71) Applicants: The United States Government As Represented By The Department Of Veterans Affairs, Washington, DC (US); The Regents of the University of California, Oakland, CA (US)

(72) Inventors: Wenhan Chang, San Francisco, CA (US); Chia-Ling Tu, San Francisco, CA (US); James Koh, San Francisco, CA (US)

(21) Appl. No.: 19/213,715

(22) Filed: May 20, 2025

Related U.S. Application Data
(60) Provisional application No. 63/649,733, filed on May 20, 2024.

(51) Int. Cl. A61K 38/08; A61K 31/277; A61K 39/395; A61P 5/20; A61P 25/28

(52) U.S. Cl. CPC A61K 38/08; A61K 31/277; A61K 39/3955; A61P 5/20; A61P 25/28

(57) ABSTRACT

Disclosed herein are methods of treating or preventing Alzheimer's disease or dementia and concurrently treating hyperparathyrodism by administering a blood-brain barrier (BBB)-impermeable calcimimetic and/or a BBB-permeable calcilytic along with the administration of anti-amyloid-beta therapies. Also disclosed herein are methods of increasing calcium-sensing receptor (CaSR) homodimer formation, increasing expression or activity of the CaSR homodimer and reducing CaSR/GABA-B1 receptor heterodimer formation, reducing expression or activity of the CaSR/GABA-B1 receptor heterodimer in both peripheral tissues and central nervous system (CNS) in a subject.

### CROSS REFERENCE TO RELATED APPLICATIONS

This application claims the benefit of the filing date of U.S. Provisional Application No. 63/649,733, filed on May 20, 2024. The content of this earlier filed application is hereby incorporated by reference in its entirety.

### STATEMENT REGARDING FEDERALLY FUNDED RESEARCH

This invention was made with government support under grant numbers BX004835, BX005851, and BX001960 awarded by United States Department of Veterans Affairs. The government has certain rights in the invention.

### BACKGROUND

Alzheimer's disease (AD) is the most common form of dementia, characterized by loss of memory, language, problem-solving, and other thinking abilities that severely interfere with daily life and imposes devastating socioeconomic burden on every community across the global. In the past decades, vast research established the “amyloid-beta (Aβ) hypothesis” that ascribes Aβ accumulation to the development and/or progression of AD. However, numerous drugs that clear Aβ in the brain have modest benefits in slowing down cognitive declines in patients, indicating additional confounders, including hyperparathyroidism (HPT) (Ilievski, V. et al. PloS One 13, e0204941 (2018); Timmons, J. G., et al. Hormones (Athens, Greece) 20, 587-589 (2021); and de Oliveira Martins Duarte, J. et al. Clin Case Rep 7, 2571-2574 (2019)), another aging-associated disease due to chronic elevation of serum parathyroid hormone (PTH) levels, which is caused by downregulation of the extracellular calcium-sensing receptor (CaSR) in the parathyroid gland (PTG). A need exists for the treatment, prevention, and management of AD and dementia

### SUMMARY OF THE INVENTION

Disclosed herein are methods of treating or preventing Alzheimer's disease or dementia in a subject, the methods comprising administering to the subject a therapeutically effective amount of a blood-brain barrier-impermeable calcimimetic or a blood-brain barrier-permeable calcilytic, thereby treating or preventing Alzheimer's disease or dementia in the subject.

Disclosed herein are methods of preventing or treating dementia in a subject, the methods comprising administering to the subject a therapeutically effective amount of a BBB-impermeable calcimimetic and an anti-Aβ therapy, thereby and preventing or treating dementia in the subject.

Disclosed herein are methods of delaying the onset or progression of dementia in a subject, the methods comprising administering to the subject a therapeutically effective amount of a blood-brain barrier-impermeable calcimimetic and an anti-amyloid-beta therapy, thereby delaying the onset or progression of dementia in the subject.

Disclosed herein are methods of delaying onset and/or progression of Alzheimer's disease in a subject, the methods comprising administering to the subject a therapeutically effective amount of a blood-brain barrier-permeable calcilytic and an anti-Aβ therapy, thereby delaying onset and/or progression of Alzheimer's disease in the subject.

Disclosed herein are methods of reducing or ameliorating one or more symptoms of Alzheimer's disease or dementia in a subject, the methods comprising administering to the subject a therapeutically effective amount of a blood-brain barrier-impermeable calcimimetic or a blood-brain barrier-permeable calcilytic.

Disclosed herein are methods of increasing calcium-sensing receptor (CaSR) homodimer formation, expression or activity in parathyroid cells and blocking CaSR/GABA-B1 receptor heterodimer formation, expression or activity in the central nervous system in a subject, the methods comprising administering to the subject a therapeutically effective amount of a blood-brain barrier (BBB)-impermeable calcimimetic and a blood-brain barrier-permeable calcilytic, thereby increasing CaSR homodimer formation, expression or activity in parathyroid cells and blocking CaSR/GABA-B1 receptor heterodimer formation, expression or activity in central nervous system in the subject.

Disclosed herein are methods of increasing calcium-sensing receptor (CaSR) homodimer activity, increasing expression or activity of a calcium-sensing receptor (CaSR) homodimer or blocking CaSR/GABA-B1 receptor heterodimer activity in both peripheral tissues and the central nervous system (CNS) in a subject, the methods comprising administering to the subject a therapeutically effective amount of a blood-brain barrier (BBB)-impermeable calcimimetic and a blood-brain barrier-permeable calcilytic, thereby increasing calcium-sensing receptor (CaSR) homodimer formation or blocking CaSR/GABA-B1 receptor heterodimer formation in peripheral tissues and the CNS, respectively, in the subject.

Disclosed herein are methods of increasing calcium-sensing receptor (CaSR) homodimer activity, increasing expression or activity of a calcium-sensing receptor (CaSR) homodimer and blocking CaSR/GABA-B1 receptor heterodimer activity in both peripheral tissues and the central nervous system (CNS) in a subject, the methods comprising administering to the subject a therapeutically effective amount of a blood-brain barrier (BBB)-impermeable calcimimetic and a blood-brain barrier-permeable calcilytic, thereby increasing calcium-sensing receptor (CaSR) homodimer formation and blocking CaSR/GABA-B1 receptor heterodimer formation in peripheral tissues and the CNS, respectively, in the subject.

Disclosed herein are methods of reducing or blocking CaSR/GABA-B1 receptor heterodimer activity in a subject, the methods comprising administering to the subject a therapeutically effective amount of a blood-brain barrier (BBB)-impermeable calcimimetic and a calcilytic, thereby reducing or blocking CaSR/GABA-B1 receptor heterodimer activity in the subject.

Disclosed herein are methods of reducing serum parathyroid hormone (PTH) levels in a subject, the methods comprising administering to the subject a therapeutically effective amount of a blood brain barrier-impermeable calcimimetic, thereby reducing serum PTH levels in the subject.

Disclosed herein are methods of reducing serum parathyroid hormone (PTH) levels without affecting CaSR activity in the CNS in a subject, the methods comprising administering to the subject a therapeutically effective amount of a blood brain barrier-impermeable calcimimetic, thereby reducing serum PTH levels without activating CaSR homodimer or CaSR/GABA-B1 receptor heterodimer in the CNS of the subject.

Disclosed herein are methods of reducing serum parathyroid hormone (PTH) levels in a subject, the methods comprising administering to the subject a therapeutically effective amount of a blood brain barrier-impermeable calcimimetic, thereby reducing serum PTH levels without enhancing adverse neuronal excitotoxicity in the subject in the subject.

Disclosed herein are methods of suppressing signaling of calcium-sensing receptor (CaSR)/GABA-B1 heterodimer in the central nervous system (CNS) in a subject, the methods comprising administering to the subject a therapeutically effective amount of an anti-amyloid-beta (anti-Aβ) therapy, thereby suppressing CaSR/GABA-B1 heterodimer signaling in the CNS and peripheral organs in the subject.

Disclosed herein are methods of reducing serum parathyroid hormone (PTH) levels in a subject, the methods comprising administering to the subject a therapeutically effective amount of anti-amyloid-beta therapy, thereby reducing serum PTH levels in the subject.

Disclosed herein are methods of reducing serum parathyroid hormone (PTH) levels in a subject, the methods comprising concurrently administering to the subject a therapeutically effective amount of a blood-brain barrier-impermeable calcimimetic and an anti-amyloid-beta therapy, thereby synergistically reducing serum PTH levels in the subject.

Disclosed herein are methods of concurrently activating calcium-sensing receptor (CaSR) homodimer signaling and blocking CaSR/GABA-B1 heterodimer signaling in a subject, the methods comprising administering to the subject a therapeutically effective amount of a blood-brain barrier-impermeable calcimimetic and an anti-Aβ therapy, thereby activating CaSR homodimer signaling and blocking CaSR/GABA-B1 heterodimer signaling, respectively, in the subject.

Disclosed herein are methods of treating hyperparathyroidism in a subject, the methods comprising administering to the subject a therapeutically effective amount of a blood-brain barrier-impermeable calcimimetic and an anti-amyloid-beta therapy, thereby suppressing parathyroid hormone secretion in the subject and thereby treating hyperparathyroidism in the subject.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1 shows the proposed actions of aging-induced hyperparathyroidism on changes in dimerization expression and signaling of calcium-sensing receptor (CaSR), GABAB1R (GBR1) or GABAB2R (GBR2) and down-stream neuronal responses and pharmaceutic regimens that target CaSR and CaSR/GABAB1R to treat Alzheimer's disease.

FIGS. 2A-D show that HPT accelerates aging-induced cognitive declines. FIGS. 2A and 2B show serum PTH (sPTH) (FIG. 2A) and calcium (sCa) (FIG. 2B) in 3 months old mixed male and female ^{PTC}VDR^{Δflox/Δflox} (^{PTC}VDR^{-/-}), ^{PTC}CaSR^{Δflox/wt} (^{PTC}CaSR^{+/-}), and control littermates (+/+) carrying floxed-VDR or floxed-CaSR alleles without PTH-Cre expression. FIGS. 2C and 2D show the spontaneous alteration index (% Spont Alt) in Y-maze tests of mixed male and female ^{PTC}VDR^{Δflox/Δflox}, ^{PTC}CaSR^{Δflox/wt}, and controls at different ages. Mean±SE, p values were derived by one-way ANOVA, n=13-46 mice/group.

FIGS. 3A-C show that daily injections of a long-acting PTH (LAPTH) cause hypercalcemia, elevated 1,25D levels, and cognitive declines. FIG. 3A show serum calcium (sCa) and 1,25D levels in the 3-month-old C57/B6 male mice at different time points following the first injection of LAPTH (40 mg/kg). Mean±SE, n=5-10 mice/time point. FIG. 3B show Spont Alt indices in Y-maze test and FIG. 3C shows discrimination indices (Disc Index, %) in NOR test of the C57/B6 male mice with once-daily (5 days/week) injections of LAPTH or Veh for 4 weeks. Control littermates at different time points. Mean±SE, p values by 2-tailed t-test, n=5 mice/group.

FIG. 4 shows HPT accelerates on-set of the early-onset AD (EOAD). Spont Alt indices in Y-maze test in 3.5 and 5-6 MOA homozygous APP^{NL-G-F} mice, modeling EOAD, with (+/−) or without (+/+) heterozygous deletion of the Casr gene in their parathyroid cells. Mean±SE, p values by one-way ANOVA, n=8-80 mixed male and female mice/group.

FIGS. 5A-F show that hypoparathyroidism delays aging-induced cognitive declines. FIGS. 5A and 5D show serum PTH, FIGS. 5B and 5E show calcium, and FIGS. 5C and 5F show Spont Alt indices in Y-maze test in mice without (+/+) or with (−/−) homozygous parathyroid cell-specific App (FIGS. 5A-C) or Gabbr1 (FIGS. 5D-F) gene KO at the months of age (MOA) as specified. Mean=SE, p values by 2-tailed t-test (FIGS. 5A, 5B, 5D, and 5E) or one-way ANOVA (FIGS. 5C and 5F), n=13-67 mixed male and female mice/group.

FIG. 6 shows that hypoparathyroidism delays on-set of EOAD. Spont Alt indices in Y-maze test in 6 MOA homozygous APP^{NL-G-F} mice with (−/−) or without (+/+) homozygous deletion of the Gabbr1 gene in their parathyroid cells. Mean±SE, p values by one-way ANOVA, n=10-28 mixed male and female mice/group.

FIG. 7 shows the effects of BBB-impermeable calcimimetics and BBB-permeable calcilytics on aging-induced cognitive declines. Spont Alt indices of Y-maze test in 5 and 18 MOA male C57/B6 mice after 8 weeks of daily (5 days/week) injections of vehicle, 500 μg/kg Etelcalcetide (Etel) and/or 12 μmole/kg NPS-2143 (2143). Mean±SE, p values by one-way ANOVA, n=6-12 mice/group.

FIG. 8 shows the effects of Etelcalcetide and NPS2143 on cognitive declines in EOAD mice. Spont Alt indices of Y-maze test in 10 MOA mice carrying humanized Ab sequence without (bAb-KI) or with (APP^{NLGF}) 4 mutations identified from EOAD patients after daily (5 days/week) injections of vehicle, 500 μg/kg Etelcalcetide (Etel) and/or 12 μmole/kg NPS-2143 (2143) beginning at 6 MOA. Mean±SE, p values by one-way ANOVA, n=19-37 mixed male and female mice/group.

FIGS. 9A-I show CaSR upregulation in postmortem brains of AD patients. Immunohistochemistry (FIG. 9A) and spatial proteomic profiling (FIGS. 9B) show increased CaSR protein expression in hippocampal neurons with the stages (0 to 6) of AD (FIG. 9C) and its correlations with several hallmarks of AD (FIGS. 9D-91). Mean±SE, p values by one-way ANOVA, n=3-6 biopsies/group.

FIG. 10 shows increased neuronal expression and co-localization of Aβ and CaSR/GABAB1R heterodimer in late stage of AD patients. Concurrent PLA to detect CaSR/GABAB1R dimers (red), fluorescence IHC to detect Ab42 (green) and NeuN (blue), and counter-staining of DNA with Syto83 (magenta) were performed on hippocampi CA1 of human postmortem AD brain at Braak stage 1 and 6. Representative images were pseudocolored, overlayed, and digitally enlarged as shown. Arrow and arrowheads indicate NeuN-(+) and NeuN-(−) cells, respectively. n=6 samples/group.

FIGS. 11A-C show in situ quantifications of CaSR, GABAB1R, GABAB2R, and APP/Aβ signaling proteins and immunohistochemical (IHC) localization of CaSR in hippocampi of 6, 12, and 24 months old male C57/B6 mice. FIGS. 11A-B show FFPE brains sections (4 mice/age group) were hybridized with a mixture of 4 sets of photocleavable barcoded-antibody probes (1. Neural Cell Profiling Core; 2. AD Pathology Module; 3. AD Pathology Extended Module; 4. a custom CaSR, GABAB1R and GABAB2R module for a total of 46 protein targets, including 3 housekeeping proteins for normalization), along with 3 morphology probes [(1. Syto-13 dye (in blue) for DNA; 2. Texas Red-IBA1 antibody (in green); 3. Cy5-NeuN (in red) antibody] for spatial tissue segregation according to manufacturer's protocol. FIG. 11A shows representative regions of interest (ROIs) in 6 and 24 months old Hipp sections with or without application of NeuN (+) segregation mask (in cyan) to define the areas to be quantified. Within the masked areas, a high-resolution (1 μm) laser was applied to release specific nucleotide barcodes from the antibody probes, which were then collected by an automated microfluidics system. The collected nucleotide barcodes were counted in an nCounter Max system and analyzed. A total of 6 ROIs [CA4, CA3, CA2, proximal CA1 (P.CA1), and 2 distal CA1 (D.CA1)] from each section were analyzed. Bottom panels show digitally enlarges ROIs at P.CA1. FIG. 11B shows a representative heatmap showing quantitative fold changes (from arithmetic mean) of 14 proteins in Aβ and tau pathways after normalization to the area of ROI and house-keeping proteins. FIG. 11C shows qualitative IHC analyses with anti-CaSR anti-serum (in red) show progressive increases in receptor expression with age. N=4 mice/age.

FIG. 12 shows immunohistochemical detection of CaSR (in green) in HIPP of 6-month-old Cont, 5×FAD, and 5×FAD+CaSR-KO mice with rabbit polyclonal antisera (VA609) against an N-terminal of the receptor. N=4 mice/group.

FIG. 13 shows increased neuronal CaSR and Aβ expression and their co-localization in the hippocampus of fAD mice. Dual fluorescence IHC was performed on hippocampal CA1 of the APP^{NLGF/NLGF} and control littermates at 12 MOA with AF488-conjuated anti-Ab₄₂ (mAducanumab; green) and AF647-conjugated anti-nCaSR (red). White arrowheads indicate extracellular AbP. Representative images (63×) were pseudocolored, overlayed (in yellow), and digitally enlarged as shown. n=4 male mice/group.

FIGS. 14A-F show expression of CaSR (FIGS. 14A-B) by immunohistochemistry and GABAB1R/GABAB2R heterodimers (FIGS. 14C-D) by proximity ligation assay in HIPP of 10-month-old APP^{NL-G-F/NL-G-F} vs control hAb-KI mice with (FIGS. 14A-D) or without (FIGS. 14E-F) Abs. N=3 mice/group.

FIG. 15 shows the expression of CaSR assessed by immunohistochemistry (left panels) and heterodimerization assessed by PLA of CaSR/GABAB1R (middle panels) and GABAB1R/GABAB2R (right panels) heterodimers in cultures of iPSC-derived neurons infected with empty lentiviral construct (Lenti-Vect) or the one expressing CaSR cDNA (Lenti-CaSR). Representative images of cultures in triplicates from 2 independent experiment.

FIGS. 16A-H show suppressing neuronal CaSR activities prevents cognitive declines. Y-maze (FIGS. 16A, 16C, 16E, 16G and 16H) and/or new object recognition (NOR) (FIGS. 14B, 14D, and 14F) tests were performed on (FIG. 16A and 16B) ^{CamK}CaSR KO mice and control littermates (+/+), which carry floxed-CaSR alleles without CamK2a promoter-driven Cre expression, at the specified months of age (MOA); FIGS. 16C-16F show neurobehavioral changes in homozygous 5×FAD mice and non-transgenic controls (Cont) with or without being bred into ^{CamK}CASR KO background (FIGS. 16C and 16D) or subjected to daily (5 days/week) subcutaneous injections of 20 μmole/kg NPS-2143 (FIGS.

16E and 16F) at 6MOA. FIGS. 16G and 16H show neurobehavioral changes in homozygous APP^{NL-G-F} mice, which carry humanized APP sequency containing 4 mutations identified from EOAD patients, and controls (Cont) with or without being bred into ^{CamK}CASR KO background (FIG. 14G) or subjected to daily (5 days/week) subcutaneous injections of 20 μmole/kg NPS-2143 (FIG. 14H) at 6MOA; Mean±SE, p values were derived by one-way ANOVA, n=9-46 mice/group.

FIG. 17 shows increased expression of Aβ₄₂ in PTGs of ageing mice. Immunohistochemical detections of endogenous Aβ₄₂ in PTGs from 18 MOA ^{PTC}App^{-/-} mice and App^{fl/fl} control littermates at 6 and 18 MOA. Aβ₄₂ immunoreactivity was visualized with Alex Fluor 488 (green). Scale bar: 125 μm.

FIG. 18 shows the ability of Aβ1-42 to stimulate PTH secretion from parathyroid glands depends on the presence of both CaSR and GABAB1R. Ex vivo assessments of the impact of different concentrations (0.3 to 1000 nM) Aβ1-42 on PTH secretion from intact PTGs cultured from the 3-month-old mice with homozygous parathyroid-specific App gene KO (square); homozygous parathyroid-specific App and Casr double gene KO (up triangle); homozygous parathyroid-specific App and Gabbr1 double gene KO (down triangle); or homozygous parathyroid-specific App, Casr, and Gabbr1 triple gene KO (diamond). Mean±s.e.m. of n mice as indicated in the plots.

FIG. 19 shows Aβ₄₂ mediated cAMP production. Averaged time courses of CAMP in normal iPSC-derived neurons natively expressing CaSR and GABAB1R and challenged by Ab₁₋₄₂. Cells were continuously perfused with buffer without or with Aβ₄₂ (horizontal bar). Data represent the mean value s.e.m. of n=20 cells and N=3 experiments.

FIG. 20 shows the ability of a murinized anti-Aβ neutralizing antibody (mAducanumab or mAdu) to suppress tonic PTH secretion, reduces serum PTH levels, and restore cognitive functions in HPT due to aging. FIG. 19A shows changes in serum PTH (sPTH), FIG. 19B shows changes in serum calcium (sCa), and FIG. 19C shows changes in neurobehavior assessed by Y-maze of male C57/B6 mice after twice-weekly injections of vehicle (brown circles: 3 MOA; black circles: 24 MOA), daily injections of Etelcalcetide (Etel, 0.3 mg/kg/day, blue triangles), aducanumab (40 mg/kg/week by twice weekly of 20 mg/kg injections, orange squares), or a combination of cinacalcet and aducanumab (purple diamonds). mean±s.e.m., of n=9-15 mice for each treatment. ns (p>0.05), **p<0.01, ***p<0.005, ****p<0.0001 by one-way ANOVA with Fisher's LSD test.

FIG. 21 shows effects of mAdu and NPS2143 on aging-indued cognitive decline. Spon Alt indices by Y-maze test were obtained from young (3MOA) and aging (24 MOA) male C57/BL6 mice with daily injections of vehicle (5 days/week), mAdu (20 mg/kg/week by 2 injections) with or without co-injection of NPS2143 (0.3 mg/kg/day, 5 days/week) for 5 weeks beginning at 23 MOA. Mean±s.e.m, p values by one-way ANOVA, n=9-14 mice/group.

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present disclosure is not entitled to antedate such publication by virtue of prior disclosures. Further, the dates of publication provided herein can be different from the actual publication dates, which can require independent confirmation.

### Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. The use of the word “a” or “an” when used in conjunction with the term “comprising” in the claims and/or the specification may mean “one,” but it is also consistent with the meaning of “one or more,” “at least one,” and “one or more than one.”

“Optional” or “optionally” means that the subsequently described event, circumstance, or material may or may not occur or be present, and that the description includes instances where the event, circumstance, or material occurs or is present and instances where it does not occur or is not present.

The word “or” as used herein means any one member of a particular list and also includes any combination of members of that list. The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the standard deviation of error for the device or method being employed to determine the value.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

“Inhibit,” “inhibiting”, “inhibition”, and “blocking” mean to diminish or decrease an activity, level, response, condition, disease, or other biological parameter. This can include, but is not limited to, the complete ablation of the activity, response, condition, or disease. This may also include, for example, a 10% inhibition or reduction in the activity, response, condition, or disease as compared to the native or control level. Thus, in some aspects, the inhibition or reduction can be a 10, 20, 30, 40, 50, 60, 70, 80, 90, 100%, or any amount of reduction in between as compared to native or control levels. In some aspects, the inhibition or reduction is 10-20, 20-30, 30-40, 40-50, 50-60, 60-70, 70-80, 80-90, or 90-100% as compared to native or control levels. In some aspects, the inhibition or reduction is 0-25, 25-50, 50-75, or 75-100% as compared to native or control levels.

“Treatment” and “treating” refer to administration or application of a therapeutic agent to a subject or performance of a procedure or modality on a subject for the purpose of obtaining a therapeutic benefit of a disease or health-related condition. For example, a treatment may include administration of a pharmaceutically effective amount of a blood-brain barrier-impermeable calcimimetic, a calcilytic, an anti-Amyloid-beta (anti-Aβ) therapy or a combination thereof.

As used herein, the term “treating” refers to partially or completely alleviating, ameliorating, relieving, delaying onset of, inhibiting or slowing progression of, reducing severity of, and/or reducing incidence of one or more symptoms or features of a particular disease, disorder, and/or condition (e.g., Alzheimer's disease, hyperparathyroidism or dementia). Treatment can be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition. For example, the disease, disorder, and/or condition can be Alzheimer's disease, hyperparathyroidism or dementia.

As used herein, the term “subject” refers to the target of administration, e.g., a human. Thus, the subject of the disclosed methods can be a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In some aspects, a subject is a mammal. In another aspect, a subject is a human. In some aspects, a subject is a non-human primate. The term does not denote a particular age or sex. Thus, adult, child, adolescent and newborn subjects, as well as fetuses, whether male or female, are intended to be covered.

As used herein, the term “patient” refers to a subject afflicted with a condition, disease or disorder (e.g., Alzheimer's disease, dementia, or hyperparathyroidism). The term “patient” includes human and veterinary subjects. In some aspects of the disclosed methods, the “patient” has been diagnosed with Alzheimer's disease, dementia, or hyperparathyroidism. In some aspects of the disclosed methods, the “patient” has been diagnosed with a need for treatment (e.g. treatment for Alzheimer's disease, dementia, or hyperparathyroidism), such as, for example, prior to the administering step.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Alzheimer's disease is the most common type of dementia. It is a progressive disease beginning with mild memory loss and other mental functions. Alzheimer's disease involves parts of the brain that control thought, memory, and language. No cure exists, but currently available medications and management strategies may temporarily improve symptoms.

Dementia is a group of conditions characterized by impairment of at least two brain functions, such as memory loss and judgment. Symptoms include forgetfulness, limited social skills, and thinking abilities become impaired such that it interferes with daily functioning. Currently available medications and therapies may help manage symptoms.

Hyperparathyroidism is when the parathyroid gland produce too much parathyroid hormone. The most common symptoms of hyperparathyroidism are chronic fatigue, body aches, difficulty sleeping, bone pain, memory loss, poor concentration, depression, and headaches. Parathyroid disease also frequently leads to osteoporosis, kidney stones, hypertension, cardiac arrhythmias, and kidney failure.

### Methods of Treatment

Disclosed herein are methods of treating or preventing Alzheimer's disease or dementia in a subject, the methods comprising administering to the subject a therapeutically effective amount of a blood-brain barrier (BBB)-impermeable calcimimetic or a blood-brain barrier (BBB)-permeable calcilytic, thereby treating or preventing Alzheimer's disease or dementia in the subject. In some aspects, the therapeutically effective amount of a blood-brain barrier (BBB)-impermeable calcimimetic increases CaSR homodimer formation, expression or activity in the parathyroid cells in the subject. In some aspects, the therapeutically effective amount of a blood-brain barrier (BBB)-impermeable calcimimetic reduces circulating PTH levels. In some aspects, the calcilytic can be a blood-brain barrier (BBB)-permeable calcilytic. In some aspects, the BBB-permeable calcilytic can be delivered systemically. In some aspects, the calcilytic can be a blood-brain barrier (BBB)-impermeable calcilytic, and can be administered directly to the central nervous system. In some aspects, the therapeutically effective amount of a blood-brain barrier-permeable calcilytic reduces CaSR/GABA-B1 receptor heterodimer formation, expression or activity in the CNS in the subject. In some aspects, the therapeutically effective amount of a blood-brain barrier-permeable calcilytic suppresses neuronal CaSR overactivity. In some aspects, the method can further comprise administering to the subject an anti-amyloid-beta (anti-Aβ) therapy. In some aspects, the administration of the anti-amyloid-beta therapy reduces beta-amyloid synthesis or circulating or tissue beta amyloid levels in the subject. In some aspects, the method can further comprise administering a therapeutically effective amount of a beta-amyloid synthesis inhibitor to the subject. In some aspects, the beta-amyloid synthesis inhibitor can be a neutralizing antibody. In some aspects, the beta-amyloid synthesis is reduced in neurons, in parathyroid cells, or both. In some aspects, the method reduces beta-amyloid plaque formation, phosphorylated tau, microglia activation or a combination thereof in the subject. In some aspects, the anti-Aβ therapy can be lecanemab (Leqembi®), aducanumab (Aduhelm®), or donanemab. In some aspects, the lecanemab (Leqembi®), aducanumab (Aduhelm®), or donanemab can be administered intravenously. In some aspects, the method reduces amyloid plaque formation in the subject. In some aspects, the method can further comprise administering a therapeutically effective amount of aducanumab-avwa to the subject. In some aspects, the aducanumab-avwa can be administered intravenously. In some aspects, the calcimimetic can be etelcalcetide. In some aspects, the calcimimetic can be administered orally, intravenously, or subcutaneously. In some aspects, the calcilytic can be NPS2143, ATF-936, AXT-914, CLTX-305, or a combination thereof. In some aspects, the calcilytic can administered orally, intravenously, subcutaneously, or intracranially. In some aspects, the subject can be a human patient. In some aspects, the subject has or is at risk for having Alzheimer's disease, dementia, stroke or a trauma-induced neuronal injury.

Disclosed herein are methods of treating or preventing Alzheimer's disease or dementia and concurrently treating hyperparathyroidism in a subject, the methods comprising administering to the subject a therapeutically effective amount of a blood-brain barrier (BBB)-impermeable calcimimetic and/or a blood-brain barrier (BBB)-permeable calcilytic, thereby treating or preventing Alzheimer's disease or dementia and concurrently treating hyperparathyroidism in the subject. In some aspects, the therapeutically effective amount of a blood-brain barrier (BBB)-impermeable calcimimetic increases CaSR homodimer formation, expression or activity in the parathyroid cells in the subject. In some aspects, the therapeutically effective amount of a blood-brain barrier (BBB)-impermeable calcimimetic reduces circulating PTH levels. In some aspects, the calcilytic can be a blood-brain barrier (BBB)-permeable calcilytic. In some aspects, the BBB-permeable calcilytic can be delivered systemically. In some aspects, the calcilytic can be a blood-brain barrier (BBB)-impermeable calcilytic, and can be administered directly to the central nervous system. In some aspects, the therapeutically effective amount of a blood-brain barrier-permeable calcilytic reduces CaSR/GABA-B1 receptor heterodimer formation, expression or activity in the CNS in the subject. In some aspects, the therapeutically effective amount of a blood-brain barrier-permeable calcilytic suppresses neuronal CaSR overactivity. In some aspects, the methods can comprise administering to the subject an anti-amyloid-beta (anti-Aβ) therapy. In some aspects, the administration of the anti-amyloid-beta therapy reduces beta-amyloid synthesis or circulating or tissue beta amyloid levels in the subject. In some aspects, the method can further comprise administering a therapeutically effective amount of a beta-amyloid synthesis inhibitor to the subject. In some aspects, the beta-amyloid synthesis inhibitor can be a neutralizing antibody. In some aspects, the beta-amyloid synthesis is reduced in neurons, in parathyroid cells, or both. In some aspects, the method reduces beta-amyloid plaque formation, phosphorylated tau, microglia activation or a combination thereof in the subject. In some aspects, the anti-Aß therapy can be lecanemab (Leqembi®), aducanumab (Aduhelm®), or donanemab. In some aspects, the lecanemab (Leqembi®), aducanumab (Aduhelm®), or donanemab can be administered intravenously. In some aspects, the method reduces amyloid plaque formation in the subject. In some aspects, the method can further comprise administering a therapeutically effective amount of aducanumab-avwa to the subject. In some aspects, the aducanumab-avwa can be administered intravenously. In some aspects, the calcimimetic can be etelcalcetide. In some aspects, the calcimimetic can be administered orally, intravenously, or subcutaneously. In some aspects, the calcilytic can be NPS2143, ATF-936, AXT-914, CLTX-305, or a combination thereof. In some aspects, the calcilytic can administered orally, intravenously, subcutaneously, or intracranially. In some aspects, the subject can be a human patient. In some aspects, the subject has or is at risk for having Alzheimer's disease, dementia, hyperthyroidism, stroke or a trauma-induced neuronal injury.

Disclosed herein are methods of increasing calcium-sensing receptor (CaSR) homodimer formation, expression or activity in parathyroid cells and blocking CaSR/GABA-B1 receptor heterodimer formation, expression or activity in the central nervous system (CNS) in a subject, the methods comprising administering to the subject a therapeutically effective amount of a blood-brain barrier (BBB)-impermeable calcimimetic and a calcilytic, thereby increasing calcium-sensing receptor (CaSR) homodimer formation, expression or activity in parathyroid cells and blocking CaSR/GABA-B1 receptor heterodimer formation in the subject. In some aspects, calcium-sensing receptor (CaSR) homodimer formation, expression or activity of the CaSR homodimer is increased and CaSR/GABA-B1 receptor heterodimer formation is blocked in both peripheral tissues and in the central nervous system. In some aspects, the increasing of CaSR homodimer formation, the increasing of the expression or activity of the CaSR homodimer occurs in peripheral tissues of the subject concurrently with the blocking of the CaSR/GABA-B1 receptor heterodimer formation in the CNS in the subject.

Disclosed herein are methods of increasing calcium-sensing receptor (CaSR) homodimer formation in a subject, the methods comprising administering to the subject a therapeutically effective amount of a blood-brain barrier (BBB)-impermeable calcimimetic and a calcilytic, thereby increasing calcium-sensing receptor (CaSR) homodimer formation in the subject. In some aspects, the increase in calcium-sensing receptor (CaSR) homodimer formation in the subject is in parathyroid cells in the subject. In some aspects, the disclosed methods can block the activity of CaSR/GABS-B1 in both parathyroid cells and the CNS of the subject.

Disclosed herein are methods of increasing calcium-sensing receptor (CaSR) expression or activity in in a subject, the methods comprising administering to the subject a therapeutically effective amount of a blood-brain barrier (BBB)-impermeable calcimimetic and a calcilytic, thereby increasing calcium-sensing receptor (CaSR) homodimer expression or activity in the subject. In some aspects, the increase in calcium-sensing receptor (CaSR) expression or activity in the subject is in parathyroid cells in the subject. In some aspects, the disclosed methods can block the activity of CaSR/GABS-B1 in both parathyroid cells and the CNS of the subject.

Disclosed herein are methods of increasing calcium-sensing receptor (CaSR) homodimer formation, increasing expression or activity of a calcium-sensing receptor (CaSR) homodimer or blocking CaSR/GABA-B1 receptor heterodimer formation in both peripheral tissues and the central nervous system (CNS) in a subject. In some aspects, the methods can comprise administering to the subject a therapeutically effective amount of a blood-brain barrier (BBB)-impermeable calcimimetic and a calcilytic, thereby increasing calcium-sensing receptor (CaSR) homodimer formation, increasing expression or activity of CaSR homodimer or blocking CaSR/GABA-B1 receptor heterodimer formation in peripheral tissues and the CNS, respectively, in the subject. In some aspects, calcium-sensing receptor (CaSR) homodimer formation, expression or activity of the CaSR homodimer is increased and CaSR/GABA-B1 receptor heterodimer formation is blocked in both peripheral tissues and in the central nervous system.

Disclosed herein are methods of increasing calcium-sensing receptor (CaSR) homodimer activity, increasing expression or activity of a calcium-sensing receptor (CaSR) homodimer and blocking CaSR/GABA-B1 receptor heterodimer activity in both peripheral tissues and the central nervous system (CNS) in a subject. In some aspects, the methods can comprise administering to the subject a therapeutically effective amount of a blood-brain barrier (BBB)-impermeable calcimimetic and a blood-brain barrier-permeable calcilytic, thereby increasing calcium-sensing receptor (CaSR) homodimer formation and blocking CaSR/GABA-B1 receptor heterodimer formation in peripheral tissues and the CNS, respectively, in the subject. In some aspects, calcium-sensing receptor (CaSR) homodimer formation, expression or activity of the CaSR homodimer is increased and CaSR/GABA-B1 receptor heterodimer formation is blocked in both peripheral tissues and in the central nervous system.

Disclosed herein are methods of increasing calcium-sensing receptor (CaSR) homodimer formation in both peripheral tissues and the central nervous system (CNS) in a subject. In some aspects, the methods can comprise administering to the subject a therapeutically effective amount of a blood-brain barrier (BBB)-impermeable calcimimetic and a calcilytic, thereby increasing calcium-sensing receptor (CaSR) homodimer formation in peripheral tissues and the CNS, respectively in the subject.

Disclosed herein are methods of increasing expression or activity of a calcium-sensing receptor (CaSR) homodimer in both peripheral tissues and the central nervous system (CNS) in a subject. In some aspects, the methods can comprise administering to the subject a therapeutically effective amount of a blood-brain barrier (BBB)-impermeable calcimimetic and a calcilytic, thereby increasing expression or activity of CaSR homodimer in peripheral tissues and the CNS.

Disclosed herein are methods of blocking CaSR/GABA-B1 receptor heterodimer formation in both peripheral tissues and the central nervous system (CNS) in a subject. In some aspects, the methods can comprise administering to the subject a therapeutically effective amount of a blood-brain barrier (BBB)-impermeable calcimimetic and a calcilytic, thereby blocking CaSR/GABA-B1 receptor heterodimer formation in peripheral tissues and the CNS.

Disclosed herein are methods of blocking CaSR/GABA-B1 receptor heterodimer formation, expression or activity in a subject, the methods comprising administering to the subject a therapeutically effective amount of a blood-brain barrier (BBB)-impermeable calcimimetic and a calcilytic, thereby blocking CaSR/GABA-B1 receptor heterodimer formation in the subject. In some aspects, the blocking of the CaSR/GABA-B1 receptor heterodimer formation, expression or activity in the subject is in central nervous system (CNS) of the subject. In some aspects, the disclosed methods can block the activity of CaSR/GABS-B1 in both parathyroid cells and the CNS of the subject.

Disclosed herein are method of reducing or blocking CaSR/GABA-B1 receptor heterodimer activity in a subject. In some aspects, the methods can comprise administering to the subject a therapeutically effective amount of a blood-brain barrier (BBB)-impermeable calcimimetic and a calcilytic, thereby reducing or blocking CaSR/GABA-B1 receptor heterodimer activity in the subject. In some aspects, the CaSR/GABA-B1 receptor heterodimer activity can be reduced or blocked in both peripheral tissues and in the central nervous system.

In some aspects, the disclosed methods can block the activity of CaSR/GABS-B1 in both parathyroid cells and the CNS of the subject. In some aspects, calcium-sensing receptor (CaSR) homodimer formation, expression or activity is increased and CaSR/GABA-B1 receptor heterodimer formation is blocked in both peripheral tissues and in the central nervous system. In some aspects, the therapeutically effective amount of a blood-brain barrier (BBB)-impermeable calcimimetic increases CaSR homodimer formation, expression or activity in peripheral tissues in the subject. In some aspects, the calcilytic is blood-brain barrier (BBB)-permeable calcilytic. In some aspects, the therapeutically effective amount of a blood-brain barrier-permeable calcilytic reduces CaSR/GABA-B1 receptor heterodimer formation, expression or activity in the CNS in the subject. In some aspects, the increasing of CaSR homodimer formation, expression or activity occurs in peripheral tissues (e.g., parathyroid glands or parathyroid cells) at the same time, simultaneously or overlaps with the reducing of CaSR/GABA-B1 receptor heterodimer formation, expression or activity in the CNS in the subject. In some aspects, the method can further comprise administering to the subject an anti-amyloid-beta (anti-Aβ) therapy. In some aspects, the method reduces beta-amyloid plaque formation, phosphorylated tau, microglia activation or a combination thereof in the subject. In some aspects, the administration of the anti-amyloid-beta therapy reduces beta-amyloid synthesis or circulating or tissue beta amyloid levels in the subject. In some aspects, the beta-amyloid synthesis or levels can be reduced in neurons, in parathyroid cells, or both. In some aspects, the method can further comprise administering a therapeutically effective amount of a beta-amyloid synthesis inhibitor to the subject. In some aspects, the beta-amyloid synthesis inhibitor can be a neutralizing antibody. In some aspects, the anti-Aβ therapy can be lecanemab (Leqembi®), aducanumab (Aduhelm®), or donanemab. In some aspects, the lecanemab (Leqembi®), aducanumab (Aduhelm®), or donanemab can be administered intravenously. In some aspects, the method reduces amyloid plaque formation in the subject. In some aspects, the method can further comprise administering a therapeutically effective amount of aducanumab-avwa to the subject. In some aspects, the aducanumab-avwa can be administered intravenously. In some aspects, the calcimimetic can be etelcalcetide. In some aspects, the calcimimetic can be administered orally, intravenously, or subcutaneously. In some aspects, the calcilytic can be NPS2143, ATF-936, AXT-914, CLTX-305, or a combination thereof. In some aspects, the calcilytic can be administered orally, intravenously, or subcutaneously. In some aspects, the subject can be a human patient. In some aspects, the subject has or is at risk for having Alzheimer's disease, dementia, stroke or a trauma-induced neuronal injury.

Disclosed herein are methods of increasing calcium-sensing receptor (CaSR) homodimer formation, expression or activity and reducing or blocking CaSR/GABA-B1 receptor heterodimer activity, expression or activity in both peripheral tissues and central nervous system (CNS) in a subject, the methods comprising administering to the subject a therapeutically effective amount of a blood-brain barrier (BBB)-impermeable calcimimetic and a calcilytic, thereby increasing CaSR homodimer formation, expression or activity and blocking or reducing CaSR/GABA-B1 receptor heterodimer activity in the subject. In some aspects, calcium-sensing receptor (CaSR) homodimer formation, expression or activity is increased and CaSR/GABA-B1 receptor heterodimer activity is reduced in both peripheral tissues and in the central nervous system. In some aspects, the calcilytic can be blood-brain barrier (BBB)-permeable calcilytic. In some aspects, the method can further comprise administering to the subject an anti-amyloid-beta (anti-Aβ) therapy. In some aspects, the method reduces beta-amyloid plaque formation, phosphorylated tau, microglia activation or a combination thereof in the subject. In some aspects, the administration of the anti-amyloid-beta therapy reduces beta-amyloid synthesis or circulating or tissue beta amyloid levels in the subject. In some aspects, the beta-amyloid synthesis or levels can be reduced in neurons, in parathyroid cells, or both. In some aspects, the method can further comprise administering a therapeutically effective amount of a beta-amyloid synthesis inhibitor to the subject. In some aspects, the beta-amyloid synthesis inhibitor can be a neutralizing antibody. In some aspects, the anti-Aβ therapy can be lecanemab (Leqembi®), aducanumab (Aduhelm®), or donanemab. In some aspects, the lecanemab (Leqembi®), aducanumab (Aduhelm®), or donanemab can be administered intravenously. In some aspects, the method reduces amyloid plaque formation in the subject. In some aspects, the method can further comprise administering a therapeutically effective amount of aducanumab-avwa to the subject. In some aspects, the aducanumab-avwa can be administered intravenously. In some aspects, the calcimimetic can be etelcalcetide. In some aspects, the calcimimetic can be administered orally, intravenously, or subcutaneously. In some aspects, the calcilytic can be NPS2143, ATF-936, AXT-914, CLTX-305, or a combination thereof. In some aspects, the calcilytic can be administered orally, intravenously, or subcutaneously. In some aspects, the subject can be a human patient. In some aspects, the subject has or is at risk for having Alzheimer's disease, dementia, stroke or a trauma-induced neuronal injury.

Disclosed herein are methods of reducing serum parathyroid hormone (PTH) levels in a subject, the methods comprising administering to the subject a therapeutically effective amount of a blood brain barrier-impermeable calcimimetic, thereby reducing serum PTH levels without enhancing adverse neuronal excitotoxicity in the subject. In some aspects, the therapeutically effective amount of a BBB-impermeable calcimimetic activates CaSR homodimer in parathyroid glands (PTGs) without activating CaSR/GABA-B1 heterodimer in CNS. In some aspects, the serum PTH levels in the subject are reduced in the subject without enhancing adverse neuronal excitotoxicity. In some aspects, the method can further comprise administering to the subject an anti-amyloid-beta (anti-Aβ) therapy. In some aspects, the method reduces beta-amyloid plaque formation, phosphorylated tau, microglia activation or a combination thereof in the subject. In some aspects, the administration of the anti-amyloid-beta therapy reduces beta-amyloid synthesis or circulating or tissue beta amyloid levels in the subject. In some aspects, the beta-amyloid synthesis or levels can be reduced in neurons, in parathyroid cells, or both. In some aspects, the method can further comprise administering a therapeutically effective amount of a beta-amyloid synthesis inhibitor to the subject. In some aspects, the beta-amyloid synthesis inhibitor can be a neutralizing antibody. In some aspects, the anti-Aβ therapy can be lecanemab (Leqembi®), aducanumab (Aduhelm®), or donanemab. In some aspects, the lecanemab (Leqembi®), aducanumab (Aduhelm®), or donanemab can be administered intravenously. In some aspects, the method reduces amyloid plaque formation in the subject. In some aspects, the method can further comprise administering a therapeutically effective amount of aducanumab-avwa to the subject. In some aspects, the aducanumab-avwa can be administered intravenously. In some aspects, the calcimimetic can be etelcalcetide. In some aspects, the calcimimetic can be administered orally, intravenously, or subcutaneously. In some aspects, the subject can be a human patient. In some aspects, the subject has or is at risk for having Alzheimer's disease, dementia, stroke or a trauma-induced neuronal injury.

Disclosed herein are methods of suppressing signaling of a calcium-sensing receptor (CaSR)/GABA-B1 heterodimer in the central nervous system and peripheral organs in a subject, the methods comprising administering to the subject a therapeutically effective amount of anti-amyloid-beta (anti-Aβ) therapy, thereby suppressing CaSR/GABA-B1 heterodimer signaling in the central nervous system and the peripheral organs in the subject. In some aspects, the method reduces beta-amyloid plaque formation, phosphorylated tau, microglia activation or a combination thereof in the subject. In some aspects, the administration of the anti-amyloid-beta therapy reduces beta-amyloid synthesis or circulating or tissue beta amyloid levels in the subject. In some aspects, the beta-amyloid synthesis or levels can be reduced in neurons, in parathyroid cells, or both. In some aspects, the method can further comprise administering a therapeutically effective amount of a beta-amyloid synthesis inhibitor to the subject. In some aspects, the beta-amyloid synthesis inhibitor can be a neutralizing antibody. In some aspects, the anti-Aβ therapy can be lecanemab (Leqembi®), aducanumab (Aduhelm®), or donanemab. In some aspects, the lecanemab (Leqembi®), aducanumab (Aduhelm®), or donanemab can be administered intravenously. In some aspects, the method reduces amyloid plaque formation in the subject. In some aspects, the method can further comprise administering a therapeutically effective amount of aducanumab-avwa to the subject. In some aspects, the aducanumab-avwa can be administered intravenously. In some aspects, the subject can be a human patient. In some aspects, the subject has or is at risk for having Alzheimer's disease, dementia, stroke or a trauma-induced neuronal injury.

Disclosed herein are methods of reducing serum parathyroid hormone (PTH) levels in a subject, the methods comprising administering to the subject a therapeutically effective amount of anti-amyloid-beta (anti-Aβ) therapy, thereby reducing serum PTH levels in the subject. In some aspects, the administration of the anti-amyloid-beta therapy reduces beta-amyloid synthesis or circulating or tissue beta amyloid levels in the subject. In some aspects, the beta-amyloid synthesis or levels can be reduced in neurons, in parathyroid cells, or both. In some aspects, the method can further comprise administering a therapeutically effective amount of a beta-amyloid synthesis inhibitor to the subject. In some aspects, the beta-amyloid synthesis inhibitor can be a neutralizing antibody. In some aspects, the anti-Aβ therapy can be lecanemab (Leqembi®), aducanumab (Aduhelm®), or donanemab. In some aspects, the lecanemab (Leqembi®), aducanumab (Aduhelm®), or donanemab can be administered intravenously. In some aspects, the method reduces amyloid plaque formation in the subject. In some aspects, the methods can further comprise administering a therapeutically effective amount of aducanumab-avwa to the subject. In some aspects, the aducanumab-avwa can be administered intravenously. In some aspects, the subject can be a human patient. In some aspects, the subject has or is at risk for having Alzheimer's disease, dementia, stroke or a trauma-induced neuronal injury.

Disclosed herein are methods of reducing serum parathyroid hormone (PTH) levels in a subject, the methods comprising concurrently administering to the subject a therapeutically effective amount of a blood-brain barrier (BBB)-impermeable calcimimetic and an anti-amyloid-beta (anti-Aβ) therapy, thereby reducing serum PTH levels in the subject. In some aspects, the method reduces beta-amyloid plaque formation, phosphorylated tau, microglia activation or a combination thereof in the subject. In some aspects, the administration of the anti-amyloid-beta therapy reduces beta-amyloid synthesis or circulating or tissue beta amyloid levels in the subject. In some aspects, the beta-amyloid synthesis or levels can be reduced in neurons, in parathyroid cells, or both. In some aspects, the method can further comprise administering a therapeutically effective amount of a beta-amyloid synthesis inhibitor to the subject. In some aspects, the beta-amyloid synthesis inhibitor can be a neutralizing antibody. In some aspects, the anti-Aβ therapy can be lecanemab (Leqembi®), aducanumab (Aduhelm®), or donanemab. In some aspects, the lecanemab (Leqembi®), aducanumab (Aduhelm®), or donanemab can be administered intravenously. In some aspects, wherein the method reduces amyloid plaque formation in the subject. In some aspects, the method can further comprise administering a therapeutically effective amount of aducanumab-avwa to the subject. In some aspects, wherein the aducanumab-avwa can be administered intravenously. In some aspects, the calcimimetic can be etelcalcetide. In some aspects, the calcimimetic can be administered orally, intravenously, or subcutaneously. In some aspects, the subject can be a human patient. In some aspects, the subject has or is at risk for having Alzheimer's disease, dementia, stroke or a trauma-induced neuronal injury.

Disclosed herein are methods of concurrently activating calcium-sensing receptor (CaSR) homodimer signaling and blocking CaSR/GABA-B1 heterodimer signaling in a subject, the methods comprising administering to the subject a therapeutically effective amount of a blood-brain barrier (BBB)-impermeable calcimimetic and an anti-amyloid-beta (anti-Aβ) therapy, thereby activating CaSR homodimer signaling and blocking CaSR/GABA-B1 heterodimer signaling, respectively, in the subject. In some aspects, the method reduces beta-amyloid plaque formation, phosphorylated tau, microglia activation or a combination thereof in the subject. In some aspects, the administration of the anti-amyloid-beta therapy reduces beta-amyloid synthesis or circulating or tissue beta amyloid levels in the subject. In some aspects, the beta-amyloid synthesis or levels can be reduced in neurons, in parathyroid cells, or both. In some aspects, the anti-Aβ therapy can be lecanemab (Leqembi®), aducanumab (Aduhelm®), or donanemab. In some aspects, the lecanemab (Leqembi®), aducanumab (Aduhelm®), or donanemab can be administered intravenously. In some aspects, the method reduces amyloid plaque formation in the subject. In some aspects, the method can further comprise administering a therapeutically effective amount of aducanumab-avwa to the subject. In some aspects, the aducanumab-avwa can be administered intravenously. In some aspects, the calcimimetic can be etelcalcetide. In some aspects, the calcimimetic can be administered orally, intravenously, or subcutaneously. In some aspects, the subject can be a human patient. In some aspects, the subject has or is at risk for having Alzheimer's disease, dementia, stroke or a trauma-induced neuronal injury.

Disclosed herein are methods of treating hyperparathyroidism in a subject, the methods comprising administering to the subject a therapeutically effective amount of a blood-brain barrier-impermeable calcimimetic and an anti-amyloid-beta (anti-Aβ) therapy, thereby suppressing parathyroid hormone secretion in the subject and thereby treating hyperparathyroidism in the subject. In some aspects, the method reduces beta-amyloid plaque formation, phosphorylated tau, microglia activation or a combination thereof in the subject. In some aspects, the administration of the anti-amyloid-beta therapy reduces beta-amyloid synthesis or circulating or tissue beta amyloid levels in the subject. In some aspects, the beta-amyloid synthesis or levels can be reduced in neurons, in parathyroid cells, or both. In some aspects, the anti-Aβ therapy can be lecanemab (Leqembi®), aducanumab (Aduhelm®), or donanemab. In some aspects, the lecanemab (Leqembi®), aducanumab (Aduhelm®), or donanemab can be administered intravenously. In some aspects, the method reduces amyloid plaque formation in the subject. In some aspects, the method can further comprise administering a therapeutically effective amount of aducanumab-avwa to the subject. In some aspects, the aducanumab-avwa can be administered intravenously. In some aspects, the calcimimetic can be etelcalcetide. In some aspects, the calcimimetic can be administered orally, intravenously, or subcutaneously. In some aspects, the subject can be a human patient. In some aspects, the subject has or is at risk for having Alzheimer's disease, dementia, stroke or a trauma-induced neuronal injury.

Disclosed herein are methods of preventing or treating dementia in a subject, the methods comprising administering to the subject a therapeutically effective amount of a blood-brain barrier-impermeable calcimimetic and an anti-amyloid-beta (anti-Aβ) therapy, thereby preventing or treating dementia. In some aspects, a therapeutically effective amount of a BBB-impermeable calcimimetic and an anti-Aβ therapy neutralize Aβ in circulation and in the CNS. In some aspects, the method reduces beta-amyloid plaque formation, phosphorylated tau, microglia activation or a combination thereof in the subject. In some aspects, the administration of the anti-amyloid-beta therapy reduces beta-amyloid synthesis or circulating or tissue beta amyloid levels in the subject. In some aspects, the beta-amyloid synthesis or levels can be reduced in neurons, in parathyroid cells, or both. In some aspects, the method can further comprise administering a therapeutically effective amount of a beta-amyloid synthesis inhibitor to the subject. In some aspects, the beta-amyloid synthesis inhibitor can be a neutralizing antibody. In some aspects, the anti-Aβ therapy can be lecanemab (Leqembi®), aducanumab (Aduhelm®), or donanemab. In some aspects, the lecanemab (Leqembi®), aducanumab (Aduhelm®), or donanemab can be administered intravenously. In some aspects, the method reduces amyloid plaque formation in the subject. In some aspects, the method can further comprise administering a therapeutically effective amount of aducanumab-avwa to the subject. In some aspects, the aducanumab-avwa can be administered intravenously. In some aspects, the calcimimetic can be etelcalcetide. In some aspects, wherein the calcimimetic can be administered orally, intravenously, or subcutaneously. In some aspects, the subject can be a human patient. In some aspects, the subject has or is at risk for having Alzheimer's disease, dementia, stroke or a trauma-induced neuronal injury.

Disclosed herein are methods of delaying onset and/or progression of Alzheimer's disease in a subject, the methods comprising administering to the subject a therapeutically effective amount of a blood-brain barrier-permeable calcilytic and an anti-amyloid-beta therapy (anti-Aβ), thereby delaying onset and/or progression of Alzheimer's disease in the subject. In some aspects, the method reduces beta-amyloid plaque formation, phosphorylated tau, microglia activation or a combination thereof in the subject. In some aspects, the administration of the anti-amyloid-beta therapy reduces beta-amyloid synthesis or circulating or tissue beta amyloid levels in the subject. In some aspects, the beta-amyloid synthesis or levels can be reduced in neurons, in parathyroid cells, or both. In some aspects, the method can further comprise administering a therapeutically effective amount of a beta-amyloid synthesis inhibitor to the subject. In some aspects, the beta-amyloid synthesis inhibitor can be a neutralizing antibody. In some aspects, the anti-Aβ therapy can be lecanemab (Leqembi®), aducanumab (Aduhelm®), or donanemab. In some aspects, the lecanemab (Leqembi®), aducanumab (Aduhelm®), or donanemab can be administered intravenously. In some aspects, wherein the method reduces amyloid plaque formation in the subject. In some aspects, the method can further comprise administering a therapeutically effective amount of aducanumab-avwa to the subject. In some aspects, the aducanumab-avwa can be administered intravenously. In some aspects, the calcilytic can be NPS2143, ATF-936, AXT-914, CLTX-305, or a combination thereof. In some aspects, the calcilytic can be administered orally, intravenously, subcutaneously or intracranially. In some aspects, the subject can be a human patient. In some aspects, wherein the subject has or is at risk for having Alzheimer's disease, dementia, stroke or a trauma-induced neuronal injury.

Disclosed herein are methods of reducing or ameliorating one or more symptoms of Alzheimer's disease or dementia in a subject, the methods comprising administering to the subject a therapeutically effective amount of a blood-brain barrier-impermeable calcimimetic or a calcilytic. In some aspects, the methods can comprise administering to the subject a therapeutically effective amount of a blood-brain barrier-impermeable calcimimetic and a calcilytic. In some aspects, the one or more symptoms of Alzheimer's disease or dementia can be loss of cognition or memory and neurodegeneration. In some aspects, the method reduces beta-amyloid plaque formation, phosphorylated tau, microglia activation or a combination thereof in the subject. In some aspects, the method can further comprise administering a therapeutically effective amount of a beta-amyloid synthesis inhibitor to the subject. In some aspects, the beta-amyloid synthesis inhibitor can be a neutralizing antibody. In some aspects, the method reduces amyloid plaque formation in the subject. In some aspects, the method can further comprise administering a therapeutically effective amount of aducanumab-avwa to the subject. In some aspects, the aducanumab-avwa can be administered intravenously. In some aspects, the calcimimetic can be etelcalcetide. In some aspects, wherein the calcimimetic can be administered orally, intravenously, or subcutaneously. In some aspects, the calcilytic can be NPS2143, ATF-936, AXT-914, CLTX-305, or a combination thereof. In some aspects, the calcilytic can be administered orally, intravenously, subcutaneously or intracranially. In some aspects, the subject can be a human patient. In some aspects, wherein the subject has or is at risk for having Alzheimer's disease, dementia, stroke or a trauma-induced neuronal injury.

Disclosed herein are methods of treating or preventing Alzheimer's disease, dementia or hyperparathyroidism in a subject. In some aspects, the methods can comprise administering to the subject a therapeutically effective amount of a blood-brain barrier (BBB)-impermeable calcimimetic or a calcilytic. In some aspects, the methods can comprise administering to the subject a therapeutically effective amount of a blood-brain barrier (BBB)-impermeable calcimimetic and a calcilytic. In some aspects, the methods can comprise administering to the subject a therapeutically effective amount of a blood-brain barrier (BBB)-impermeable calcimimetic, a calcilytic, and an anti-amyloid beta therapy. In some aspects, the methods can comprise administering to the subject a therapeutically effective amount of a blood-brain barrier (BBB)-impermeable calcimimetic, a calcilytic, an anti-amyloid beta therapy, or a combination thereof. For example, disclosed herein are methods of treating or preventing Alzheimer's disease or dementia in a subject, the method comprising administering to the subject a therapeutically effective amount of a blood-brain barrier BBB-permeable calcilytic.

Disclosed herein are methods of preventing or treating dementia in a subject. In some aspects, the methods comprises administering to the subject a therapeutically effective amount of a blood-brain barrier-impermeable calcimimetic and an anti-amyloid-beta therapy, thereby preventing or treating dementia in the subject. In some aspects, the methods of preventing or treating dementia in a subject can be achieved by suppressing chronic parathyroid hormone (PTH) secretion that causally promotes age-induced losses of cognitive function. In some aspects, the administering to the subject the therapeutically effective amount of the blood-brain barrier-impermeable calcimimetic and the anti-amyloid-beta therapy promotes CaSR homodimer expression, activity, or formation and reduces CaSR/GABA-B1 heterodimer expression, activity, or formation in parathyroid glands to synergistically suppress PTH secretion in the subject.

Also, disclosed herein are methods of delaying the onset or progression of dementia in a subject. In some aspects, the methods comprises administering to the subject a therapeutically effective amount of a blood-brain barrier-impermeable calcimimetic and an anti-amyloid-beta therapy, thereby delaying the onset or progression of dementia in the subject. In some aspects, the methods of delaying the onset or progression of dementia in a subject can be achieved by suppressing chronic parathyroid hormone (PTH) secretion that causally promotes age-induced losses of cognitive function. In some aspects, the administering to the subject the therapeutically effective amount of the blood-brain barrier-impermeable calcimimetic and the anti-amyloid-beta therapy promotes CaSR homodimer expression, activity, or formation and reduces CaSR/GABA-B1 heterodimer expression, activity, or formation in parathyroid glands to synergistically suppress PTH secretion in the subject.

Disclosed herein are methods of delaying onset and/or progression of Alzheimer's disease in a subject. In some aspects, the methods comprise administering to the subject a therapeutically effective amount of a blood-brain barrier-impermeable calcilytic and an anti-amyloid-beta therapy, thereby delaying onset and/or progression of Alzheimer's disease in the subject. In some aspects, a therapeutically effective amount of a blood-brain barrier-impermeable calcilytic and an anti-amyloid-beta therapy synergistically suppresses CaSR/GABA-B1 heterodimer expression, formation, or activity. In some aspects, CaSR/GABA-B1 heterodimer expression, formation, or activity can cause neurodegeneration, and, thus, suppresses CaSR/GABA-B1 heterodimer expression, formation, or activity can delaying onset and/or progression of Alzheimer's disease in a subject.

Disclosed herein are methods of reducing or ameliorating one or more symptoms of Alzheimer's disease or dementia in a subject. In some aspects, the methods comprise administering to the subject a therapeutically effective amount of a blood-brain barrier-impermeable calcimimetic or a calcilytic. In some aspects, the one or more symptoms of Alzheimer's disease or dementia is loss of cognition or memory and neurodegeneration. Examples of symptoms of Alzheimer's disease or dementia include, but are not limited to, difficulties with coming up with the right word or name, remembering names when introduced to new people, having difficulty performing tasks in social or work settings, forgetting material that was just read, losing or misplacing a valuable object, experiencing increased trouble with planning or organizing, being forgetful of events or personal history, feeling moody or withdrawn, especially in socially or mentally challenging situations, being unable to recall information about themselves like their address or telephone number, and the high school or college they attended, experiencing confusion about where they are or what day it is, requiring help choosing proper clothing for the season or the occasion, having trouble controlling their bladder and bowels, experiencing changes in sleep patterns, such as sleeping during the day and becoming restless at night, showing an increased tendency to wander and become lost, demonstrating personality and behavioral changes, including suspiciousness and delusions or compulsive, repetitive behavior like hand-wringing or tissue shredding, requiring around-the-clock assistance with daily personal care, losing awareness of recent experiences as well as of their surroundings, experience changes in physical abilities, including walking, sitting and, eventually, swallowing, have difficulty communicating, and becoming vulnerable to infections (e.g., pneumonia). In some aspects, neurodegeneration can be detected by (1) laboratory testing, including blood tests of a biomarker, such as neurofilament light chain (NfL), and genetic tests of risk genes, such as APP, BACE1, APOE, and PSEN1/PSEN2 genes and/or (2) brain imaging scans, including computed tomography (CT) scans, magnetic resonance imaging (MRI scans) and other imaging tests are often very important in diagnosing these conditions.

Disclosed herein are methods of increasing calcium-sensing receptor (CaSR) homodimer formation, expression or activity; and reducing CaSR/GABA-B1 receptor heterodimer formation, expression or activity in a subject. Disclosed herein are methods of increasing calcium-sensing receptor (CaSR) homodimer formation, expression or activity in peripheral tissues and reducing CaSR/GABA-B1 receptor heterodimer formation, expression or activity in the central nervous system (CNS) in a subject. In some aspects, the method comprising administering to the subject a therapeutically effective amount of a blood-brain barrier (BBB)-impermeable calcimimetic and a calcilytic, thereby increasing CaSR homodimer formation, expression or activity; and reducing of CaSR/GABA-B1 receptor heterodimer formation, expression or activity in the subject. In some aspects, the therapeutically effective amount of a blood-brain barrier (BBB)-impermeable calcimimetic increases CaSR homodimer formation, expression or activity in peripheral tissues in the subject. In some aspects, the therapeutically effective amount of a calcilytic reduces CaSR/GABA-B1 receptor heterodimer formation, expression or activity in the CNS in the subject. In some aspects, the increasing of CaSR homodimer formation, expression or activity in peripheral tissues occurs at the same time, simultaneously or overlaps with the reducing of CaSR/GABA-B1 receptor heterodimer formation, expression or activity in the CNS in the subject.

Disclosed herein are methods of increasing calcium-sensing receptor (CaSR) homodimer activity in peripheral tissues; and reducing CaSR/GABA-B1 receptor heterodimer activity in the central nervous system (CNS) in a subject. In some aspects, the methods comprise administering to the subject a therapeutically effective amount of a blood-brain barrier-impermeable calcimimetic and a calcilytic, thereby increasing CaSR homodimer activity and reducing CaSR/GABA-B1 receptor heterodimer activity in peripheral tissues and the CNS, respectively, in the subject. In some aspects, activating calcium-sensing receptor (CaSR) homodimer formation, expression or activity in peripheral tissues and blocking CaSR/GABA-B1 receptor heterodimer formation, expression, activity occurs concurrently in peripheral tissues and in the central nervous system (CNS).

Disclosed herein are methods of concurrently activating calcium-sensing receptor (CaSR) homodimer signaling and blocking CaSR/GABA-B1 heterodimer signaling in a subject. In some aspects, the methods comprise administering to the subject a therapeutically effective amount of a blood-brain barrier-impermeable calcimimetic and an anti-Aβ therapy, thereby increasing CaSR homodimer signaling and reducing CaSR/GABA-B1 heterodimer signaling, respectively, in the subject.

Disclosed herein are methods of reducing serum parathyroid hormone (PTH) levels in a subject. In some aspects, the methods comprise administering to the subject a therapeutically effective amount of a BBB-impermeable calcimimetic, thereby reducing serum PTH levels in the subject. In some aspects, the therapeutically effective amount of a BBB-impermeable calcimimetic activates CaSR homodimer in parathyroid glands (PTGs) without activating CaSR homodimer or CaSR/GABA-B1 heterodimer in CNS. In some aspects, methods do not effect or have not effect on CaSR activity in the CNS. In some aspects, serum PTH levels in the subject are reduced in the subject without enhancing adverse neuronal excitotoxicity. In some aspects, serum PTH levels in the subject are reduced by administration of a BBB-impermeable calcimimetic to the subject without enhancing adverse neuronal excitotoxicity.

Disclosed herein are methods of suppressing CaSR/GABA-B1 heterodimer signaling in the central nervous system and peripheral organs in a subject. In some aspects, the methods comprises administering to the subject a therapeutically effective amount of an anti-amyloid-beta (anti-Aβ) therapy, thereby suppressing CaSR/GABA-B1 heterodimer signaling in central nervous system and the peripheral organs (e.g., parathyroid glands, parathyroid cells) in the subject. In some aspects, the anti-Aβ therapy clears circulating Aβ that activate CaSR/GABA-B1 signaling. In some aspects, the anti-Aβ therapy can clear circulating Aβ that activate CaSR/GABA-B1 signaling in parathyroid cells to promote PTH secretion. In some aspects, the anti-Aβ therapy can clear circulating Aβ that activate CaSR/GABA-B1 signaling in bone and/or kidney to alter calciotropic actions to prevent hypercalcemia. In some aspects, the peripheral organ can be bone. In some aspects, the peripheral organ can be a kidney. In some aspects, the peripheral organ can be one or more parathyroid glands.

Disclosed herein are methods of suppressing CaSR/GABA-B1 heterodimer signaling in the central nervous system and peripheral organs in a subject. In some aspects, the methods comprises administering to the subject a therapeutically effective amount of an anti-amyloid-beta (anti-Aβ) therapy, thereby suppressing CaSR/GABA-B1 heterodimer signaling in kidney, bone or parathyroid glands.

Disclosed herein are methods of reducing serum parathyroid hormone (PTH) levels in a subject. In some aspects, the methods comprise administering to the subject a therapeutically effective amount of anti-amyloid-beta (anti-Aβ) therapy, thereby reducing serum PTH levels in the subject. In some aspects, the method comprises concurrently administering to the subject a therapeutically effective amount of a blood-brain barrier (BBB)-impermeable calcimimetic and anti-amyloid-beta (anti-Aβ) therapy, thereby synergistically reducing serum PTH levels in the subject. In some aspects, the anti-Aβ therapy can clear local Aβ in parathyroid glands.

Disclosed herein are methods of treating hyperparathyroidism in a subject. In some aspects, the methods comprise administering to the subject a therapeutically effective amount of a blood-brain barrier-impermeable calcimimetic and an anti-amyloid-beta therapy, thereby suppressing parathyroid hormone (PTH) secretion in the subject. In some aspects, the therapeutically effective amount of the blood-brain barrier-impermeable calcimimetic and the anti-amyloid-beta therapy synergistically suppress PTH secretion in the subject. In some aspects, the therapeutically effective amount of the blood-brain barrier-impermeable calcimimetic and the anti-amyloid-beta therapy increasing the CaSR homodimer signaling and reduce CaSR/GABA-B1 heterodimer signaling to synergistically suppress PTH secretion in the subject.

In some aspects, the methods disclosed herein reduces beta-amyloid plaque formation, phosphorylated tau, microglia activation or a combination thereof in the subject. In some aspects, the methods reduce amyloid plaque formation in the subject.

In some aspects of the methods disclosed herein, a therapeutically effective amount of the blood-brain barrier-impermeable calcimimetic and the calcilytic can be administered to the subject concurrently, simultaneously or sequentially.

In some aspects of the methods disclosed herein, the calcimimetic can be etelcalcetide or GSK3004774. In some aspects, the dose can be in an amount that can reduce the serum PTH levels in the subject by at least 30% or more within 1 hr post-administration. In some aspects of the methods disclosed herein, the calcimimetic can be administered orally, intravenously, or subcutaneously. In some aspects of the methods disclosed herein, the calcimimetic activates, increases, or enhances CaSR homodimer activity, formation, or expression in peripheral tissues or organs.

In some aspects of the methods disclosed herein, the calcilytic can be NPS2143, Calhex-231, Ronacaleret, encaleret, ATF-936, AXT-914, CLTX-305, or a combination thereof. In some aspects of the methods disclosed herein, the calcilytic can be administered orally, intravenously, or subcutaneously. In some aspects, the calcilytic can be administered to the subject at a dose that can increase serum PTH levels in the subject by 30% or more within 1 hr post-administration. In some aspects of the methods disclosed herein, the calcilytic blocks, inhibits, reduces or decreases CaSR/GABA-B1 receptor heterodimer activity, formation, or expression in the central nervous system (CNS).

In some aspects, any of the methods can further comprise administering a therapeutically effective amount of a beta-amyloid synthesis inhibitor to the subject. In some aspects, the beta-amyloid synthesis inhibitor can be a neutralizing antibody. In some aspects, the beta-amyloid synthesis can be reduced in neuron, in parathyroid cells, or both.

In some aspects of the methods disclosed herein, the administration of the anti-amyloid-beta therapy can reduce beta-amyloid synthesis in the subject. In some aspects of the methods disclosed herein, the administration of the anti-amyloid-beta therapy can reduce circulating beta-amyloid levels in the subject. In some aspects of the methods disclosed herein, the administration of the anti-amyloid-beta therapy can reduce tissue beta-amyloid levels in the subject. In some aspects of the methods disclosed herein, the beta-amyloid synthesis can be reduced in neurons, in parathyroid cells, or both. In some aspects of the methods disclosed herein, the beta-amyloid levels can be reduced in neurons, in parathyroid cells, or both. In some aspects of the methods disclosed herein, the anti-amyloid-beta therapy can be lecanemab (Leqembi®), aducanumab (Aduhelm®), or donanemab. In some aspects, the lecanemab (Leqembi®), aducanumab (Aduhelm®), or donanemab can be administered intravenously. In some aspects, the anti-amyloid-beta therapy can be administered to the subject at a dose of 10-40 mg/kg over a 1 hour infusion, once every 2-4 weeks.

In some aspects, the methods can further comprise administering a therapeutically effective amount of aducanumab-avwa to the subject. In some aspects of the methods disclosed herein, the aducanumab-avwa can be administered intravenously. In some aspects, the aducanumab-avwa can be administered to the subject at a dose of 10-40 mg/kg over a 1 hour infusion, once every 2-4 weeks.

The compositions described herein can be formulated to include a therapeutically effective amount of a blood-brain barrier-impermeable calcimimetic or a blood-brain barrier-permeable calcilytic or an anti-amyloid-beta (anti-Aβ) therapy described herein. Therapeutic administration encompasses prophylactic applications (e.g., or preventing Alzheimer's disease or dementia). Based on genetic testing and other prognostic methods, a physician in consultation with their patient can choose a prophylactic administration where the patient has a clinically determined predisposition or increased susceptibility (in some cases, a greatly increased susceptibility) to Alzheimer's disease or dementia.

The compositions described herein can be administered to the subject (e.g., a human patient) in an amount sufficient to delay, reduce, or preferably prevent the onset of clinical disease. In some aspects of the methods disclosed herein, the subject has or is at risk for having Alzheimer's disease, stroke, or a trauma-induced neuronal injury. In some aspects, the trauma-induced neuronal injury can be due to penetrating or non-penetrating physical impact to the head. Accordingly, in some aspects of the methods disclosed herein, the patient can be a human patient. In therapeutic applications, compositions can be administered to a subject (e.g., a human patient) already with or diagnosed with Alzheimer's disease or dementia, increased levels or amounts of PTH in serum or blood of a subject, or one or more symptoms of Alzheimer's disease or dementia in an amount sufficient to at least partially improve a sign or symptom or to inhibit the progression of (and preferably arrest) the symptoms of the condition, its complications, and consequences. An amount adequate to accomplish this is defined as a “therapeutically effective amount.” A therapeutically effective amount of a composition (e.g., a pharmaceutical composition) can be an amount that achieves a cure, but that outcome is only one among several that can be achieved. As noted, a therapeutically effective amount includes amounts that provide a treatment in which the onset or progression of the disease, disorder, condition or injury is delayed, hindered, or prevented, or the disease, disorder, condition or injury or a symptom of the disease, disorder, condition or injury is ameliorated or its frequency can be reduced. One or more of the symptoms can be less severe. Recovery can be accelerated in an individual who has been treated. For example, treatment of Alzheimer's disease or dementia may involve, for example, increase in calcium-sensing receptor (CaSR) homodimer formation, expression or activity in peripheral tissue or organs, a reduction in CaSR/GABA-B1 receptor heterodimer formation, expression or activity in the CNS, or a reduction in serum parathyroid hormone levels.

In some aspects of the methods disclosed herein, the blood-brain barrier-impermeable calcimimetic or blood-brain barrier-permeable calcilytic can be administered with at least a second therapeutic agent. The methods and compositions, including combination therapies, can enhance the therapeutic or protective effect, and/or increase the therapeutic effect to any of the blood-brain barrier-impermeable calcimimetic or the blood-brain barrier-permeable calcilytic described herein. In some aspects of the methods disclosed herein, the second therapeutic agent can be an anti-Aβ therapy.

The blood-brain barrier-impermeable calcimimetic or the blood-brain barrier-permeable calcilytic or the anti-amyloid-beta (anti-Aβ) therapy or combination thereof can be administered before, during, after, or in various combinations relative to each other or a second therapeutic agent or therapy. The administrations may be in intervals ranging from concurrently to minutes to days to weeks. In aspects where the blood-brain barrier-impermeable calcimimetic or the blood-brain barrier-permeable calcilytic or the anti-amyloid-beta (anti-Aβ) therapy is provided to a patient separately from a second therapeutic agent or therapy, one would generally ensure that a significant period of time did not expire between the time of each delivery, such that the two compounds would still be able to exert an advantageously combined effect on the patient. In such instances, it is contemplated that one may provide a patient with the blood-brain barrier-impermeable calcimimetic or the blood-brain barrier-permeable calcilytic or the anti-amyloid-beta (anti-Aβ) therapy and the second therapeutic agent or therapy within about 12 to 24 or 72 h of each other and, more particularly, within about 6-12 h of each other. In such instances, it is contemplated that one may provide a patient with the blood-brain barrier-impermeable calcimimetic or the blood-brain barrier-permeable calcilytic or the anti-amyloid-beta (anti-Aβ) therapy and the blood-brain barrier-impermeable calcimimetic or the blood-brain barrier-permeable calcilytic or the anti-amyloid-beta (anti-Aβ) therapy within about 12 to 24 or 72 h of each other and, more particularly, within about 6-12 h of each other. In some situations, it may be desirable to extend the time period for treatment significantly where several days (2, 3, 4, 5, 6, or 7) to several weeks (1, 2, 3, 4, 5, 6, 7, or 8) lapse between respective administrations.

In some aspects of the methods disclosed herein, a course of treatment can last between 1-90 days or more (this such range includes intervening days). It is contemplated that one agent may be given on any day of day 1 to day 90 (this such range includes intervening days) or any combination thereof, and another agent is given on any day of day 1 to day 90 (this such range includes intervening days) or any combination thereof. Within a single day (24-hour period), the patient may be given one or multiple administrations of the agent(s). Moreover, after a course of treatment, it is contemplated that there can be a period of time at which no anti-cancer treatment is administered. This time period may last 1-7 days, and/or 1-5 weeks, and/or 1-12 months or more (this such range includes intervening days), depending on the condition of the patient, such as their prognosis, strength, health, etc. It is expected that the treatment cycles would be repeated as necessary.

Various combinations may be employed. For the example below the blood-brain barrier-impermeable calcimimetic or the blood-brain barrier-permeable calcilytic or the anti-amyloid-beta (anti-Aβ) therapy is “A” and a second therapeutic agent or the blood-brain barrier-impermeable calcimimetic or the blood-brain barrier-permeable calcilytic or the anti-amyloid-beta (anti-Aβ) therapy is “B”:
A/B/A B/A/B B/B/A A/A/B A/B/B B/A/A A/B/B/B B/A/B/B B/B/B/A B/B/A/B A/A/B/B A/B/A/B A/B/B/A B/B/A/A B/A/B/A B/A/A/B A/A/A/B B/A/A/A A/B/A/A A/A/B/A.

Administration of any compound or therapy disclosed herein to a patient will follow general protocols for the administration of such compounds, taking into account the toxicity, if any, of the agents. Therefore, in some aspects there can be a step of monitoring toxicity that can be attributable to combination therapy.

In some aspects of the methods disclosed herein, the second therapeutic agent can be any compound that is capable of simultaneously activating a CaSR homodimer and suppressing CaSR/GABA-B1.

The compositions described herein used in the disclosed methods can be formulated to include a therapeutically effective amount of the blood-brain barrier-impermeable calcimimetic or the blood-brain barrier-permeable calcilytic or the anti-amyloid-beta (anti-Aβ) therapy disclosed herein. In some aspects of the methods disclosed herein, the blood-brain barrier-impermeable calcimimetic or the blood-brain barrier-permeable calcilytic or the anti-amyloid-beta (anti-Aβ) therapy disclosed herein can be contained within a pharmaceutical formulation. In some aspects of the methods disclosed herein, the pharmaceutical formulation can be a unit dosage formulation.

The therapeutically effective amount or dosage of any of the blood-brain barrier-impermeable calcimimetics or the calcilytics or the anti-amyloid-beta (anti-Aβ) therapies used in the methods as disclosed herein applied to mammals (e.g., humans) can be determined by one of ordinary skill in the art with consideration of individual differences in age, weight, sex, the severity of the subject's symptoms, and the particular composition or route of administration selected, other drugs administered and the judgment of the attending clinician. Variations in the needed dosage may be expected. Variations in dosage levels can be adjusted using standard empirical routes for optimization. The particular dosage of a pharmaceutical composition to be administered to the patient will depend on a variety of considerations (e.g., the severity of the symptoms), the age and physical characteristics of the subject and other considerations known to those of ordinary skill in the art. Dosages can be established using clinical approaches known to one of ordinary skill in the art. A therapeutically effective dosage of the blood-brain barrier-impermeable calcimimetic or the blood-brain barrier-permeable calcilytic or the anti-amyloid-beta (anti-Aβ) therapy can result in a decrease in severity of one or more disease symptoms, an increase in frequency and duration of disease symptom-free periods, or a prevention of impairment or disability due to the disease affliction. As disclosed therein, in some aspects a therapeutically effective amount of the blood-brain barrier-impermeable calcimimetic or the blood-brain barrier-permeable calcilytic or the anti-amyloid-beta (anti-Aβ) therapy can increase in calcium-sensing receptor (CaSR) homodimer formation, expression or activity in peripheral tissue or organs, a reduction in CaSR/GABA-B1 receptor heterodimer formation, expression or activity in the CNS, a reduction in serum parathyroid hormone levels, or otherwise reduce or ameliorate one or more symptoms in a subject.

The duration of treatment with any composition in the methods disclosed herein can be any length of time from as short as one day to as long as the life span of the host (e.g., many years). For example, the compositions can be administered once a week (for, for example, 4 weeks to many months or years); once a month (for, for example, three to twelve months or for many years); or once a year for a period of 5 years, ten years, or longer. It is also noted that the frequency of treatment can be variable. For example, the present compositions can be administered once (or twice, three times, etc.) daily, weekly, monthly, or yearly.

The total effective amount of the blood-brain barrier-impermeable calcimimetic or the calcilytic or the anti-amyloid-beta (anti-Aβ) therapy as disclosed herein can be administered to a subject as a single dose, either as a bolus or by infusion over a relatively short period of time, or can be administered using a fractionated treatment protocol in which multiple doses are administered over a more prolonged period of time. Alternatively, continuous intravenous infusions sufficient to maintain therapeutically effective concentrations in the blood are also within the scope of the present disclosure.

### Pharmaceutical Compositions

As disclosed herein, are pharmaceutical compositions, comprising one or more of the therapeutic compositions or the blood-brain barrier-impermeable calcimimetic or the calcilytic or the anti-amyloid-beta (anti-Aβ) therapy disclosed herein. As disclosed herein, are pharmaceutical compositions, comprising a blood-brain barrier-impermeable calcimimetic or a calcilytic or an anti-amyloid-beta (anti-Aβ) therapy and a pharmaceutical acceptable carrier described herein. In some aspects of the methods disclosed herein, the blood-brain barrier-impermeable calcimimetic or the calcilytic or the anti-amyloid-beta (anti-Aβ) therapy can be formulated for oral or parental administration. In some aspects of the methods disclosed herein, the parental administration can be intravenous, subcutaneous, intramuscular or direct injection. In some aspects of the methods disclosed herein, the blood-brain barrier-impermeable calcimimetic or the calcilytic or the anti-amyloid-beta (anti-Aβ) therapy can be administered intramuscularly, intravenously, subcutaneously, orally, topically, transdermally, or sublingually. The compositions can be formulated for administration by any of a variety of routes of administration, and can include one or more physiologically acceptable excipients, which can vary depending on the route of administration. As used herein, the term “excipient” means any compound or substance, including those that can also be referred to as “carriers” or “diluents.” Preparing pharmaceutical and physiologically acceptable compositions is considered routine in the art, and thus, one of ordinary skill in the art can consult numerous authorities for guidance if needed.

The compositions can be administered directly to a subject. Generally, the compositions can be suspended in a pharmaceutically acceptable carrier (e.g., physiological saline or a buffered saline solution) to facilitate their delivery. Encapsulation of the compositions in a suitable delivery vehicle (e.g., polymeric microparticles or implantable devices) may increase the efficiency of delivery.

The compositions can be formulated in various ways for parenteral or nonparenteral administration. Where suitable, oral formulations can take the form of tablets, pills, capsules, or powders, which may be enterically coated or otherwise protected. Sustained release formulations, suspensions, elixirs, aerosols, and the like can also be used.

Pharmaceutically acceptable carriers and excipients can be incorporated (e.g., water, saline, aqueous dextrose, and glycols, oils (including those of petroleum, animal, vegetable or synthetic origin), starch, cellulose, talc, glucose, lactose, sucrose, gelatin, malt, rice, flour, chalk, silica gel, magnesium stearate, sodium stearate, glycerol monosterate, sodium chloride, dried skim milk, glycerol, propylene glycol, ethanol, and the like). The compositions may be subjected to conventional pharmaceutical expedients such as sterilization and may contain conventional pharmaceutical additives such as preservatives, stabilizing agents, wetting or emulsifying agents, salts for adjusting osmotic pressure, buffers, and the like. Suitable pharmaceutical carriers and their formulations are described in “Remington's Pharmaceutical Sciences” by E. W. Martin, which is herein incorporated by reference. Such compositions will, in any event, contain an effective amount of the compositions together with a suitable amount of carrier so as to prepare the proper dosage form for proper administration to the patient.

The pharmaceutical compositions as disclosed herein can be prepared for oral or parenteral administration. Pharmaceutical compositions prepared for parenteral administration include those prepared for intravenous (or intra-arterial), intramuscular, subcutaneous, intraperitoneal, transmucosal (e.g., intranasal, intravaginal, or rectal), or transdermal (e.g., topical) administration. Aerosol inhalation can also be used. Thus, compositions can be prepared for parenteral administration that includes any of the blood-brain barrier-impermeable calcimimetics or the calcilytics or the anti-amyloid-beta (anti-Aβ) therapies dissolved or suspended in an acceptable carrier, including but not limited to an aqueous carrier, such as water, buffered water, saline, buffered saline (e.g., PBS), and the like. One or more of the excipients included can help approximate physiological conditions, such as pH adjusting and buffering agents, tonicity adjusting agents, wetting agents, detergents, and the like. Where the compositions include a solid component (as they may for oral administration), one or more of the excipients can act as a binder or filler (e.g., for the formulation of a tablet, a capsule, and the like).

The pharmaceutical compositions can be sterile and sterilized by conventional sterilization techniques or sterile filtered. Aqueous solutions can be packaged for use as is, or lyophilized, the lyophilized preparation, which is encompassed by the present disclosure, can be combined with a sterile aqueous carrier prior to administration. The pH of the pharmaceutical compositions typically will be between 3 and 11 (e.g., between about 5 and 9) or between 6 and 8 (e.g., between about 7 and 8). The resulting compositions in solid form can be packaged in multiple single dose units, each containing a fixed amount of the above-mentioned agent or agents, such as in a sealed package of tablets or capsules.

### Articles of Manufacture

The composition described herein can be packaged in a suitable container labeled, for example, for use as a therapy to treating or preventing Alzheimer's disease or dementia or any of the methods disclosed herein. Accordingly, packaged products (e.g., sterile containers containing the composition described herein and packaged for storage, shipment, or sale at concentrated or ready-to-use concentrations) and kits, including at least one or more of the blood-brain barrier-impermeable calcimimetics or the calcilytics or the anti-amyloid-beta (anti-Aβ) therapies as described herein and instructions for use, are also within the scope of the disclosure. A product can include a container (e.g., a vial, jar, bottle, bag, or the like) containing the composition described herein. In addition, an article of manufacture further may include, for example, packaging materials, instructions for use, syringes, buffers or other control reagents for treating or monitoring the condition for which prophylaxis or treatment is required. The product may also include a legend (e.g., a printed label or insert or other medium describing the product's use (e.g., an audio- or videotape)). The legend can be associated with the container (e.g., affixed to the container) and can describe the manner in which the compound therein should be administered (e.g., the frequency and route of administration), indications therefor, and other uses. The compositions can be ready for administration (e.g., present in dose-appropriate units), and may include a pharmaceutically acceptable adjuvant, carrier or other diluent. Alternatively, the compositions can be provided in a concentrated form with a diluent and instructions for dilution.

### EXAMPLES

It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

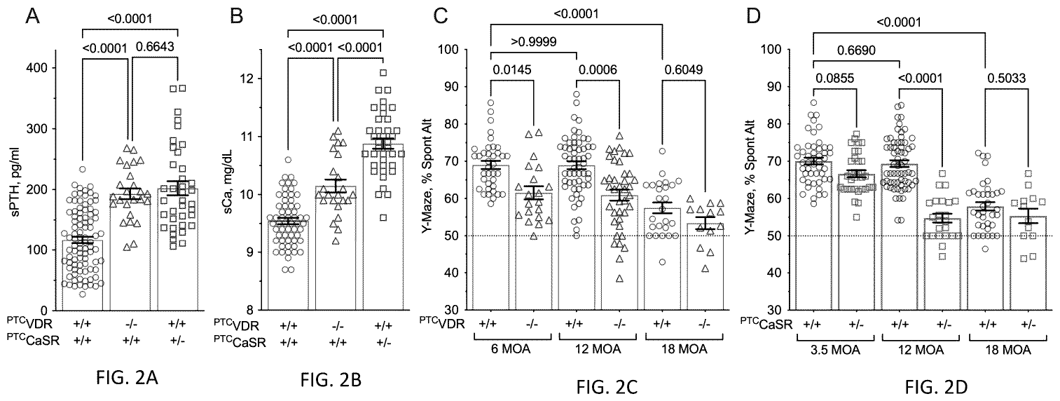

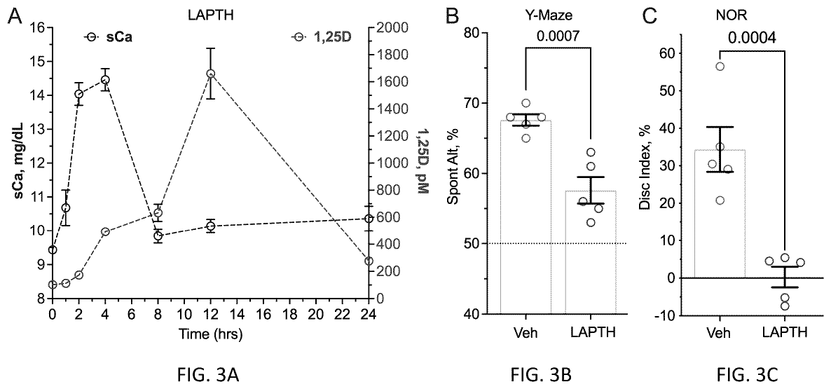

To determine whether hyperparathyroidism (HPT) plays a role in dementia, neurobehaviors were studied using three HPT models (^{PTC}CaSR^{Δflox/wt}, ^{PTC}VDR^{Δflox/Δflox}, and LAPTH-injected mice) and two hypoparathyroidism mice (^{PTC}APP1/2^{Δflox/Δflox} or ^{PTC}GABA_{B1}R^{Δflox/Δflox} mice. HPT mouse models due to parathyroid cell (PTC)-targeted ablation of the extracellular calcium-sensing receptor (CaSR) (^{PTC}CaSR^{Δflox/wt}), a family C GPCR that responds to changes in ambient calcium concentration ([Ca²⁺]), or the vitamin D receptor (VDR) (^{PTC}VDR^{Δflox/Δflox}, modeling vitamin D deficiency), show elevated serum PTH levels (FIG. 2A) and the resulting hypercalcemia (FIG. 2B) as early as 3 months of age. While in their respective control littermates (+/+), aging did not affect cognitive functions assessed by the Y-maze test until 18 months of age (18 MOA) (FIGS. 2C,2D, open circles), HPT could modestly cause cognitive declines as early as 6 and 3.5 MOA in the ^{PTC}VDR^{Δflox/Δflox} (FIG. 2C, blue triangles) and ^{PTC}CaSR^{Δflox/wt} (FIG. 2D, red squares) mice, respectively, and much more severely at 12 MOA. A pharmacological approach was carried out to corroborate the genetic studies described herein by daily injections (5 days/week) of a long-acting PTH analog (LAPTH) in C57/B6 mice, beginning at 3 MOA. The LAPTH produces severe hypercalcemia and elevated 1,25-dihydroxyvitamin D (1,25D) levels that sustained for more than 4 and 12 hrs, respectively, after each injection (FIG. 3A). After 4 weeks of injection, LAPTH caused cognitive declines, assessed by Y-maze (FIG. 3B) and NOR (FIG. 3C) tests, compared to vehicle (Veh) controls.

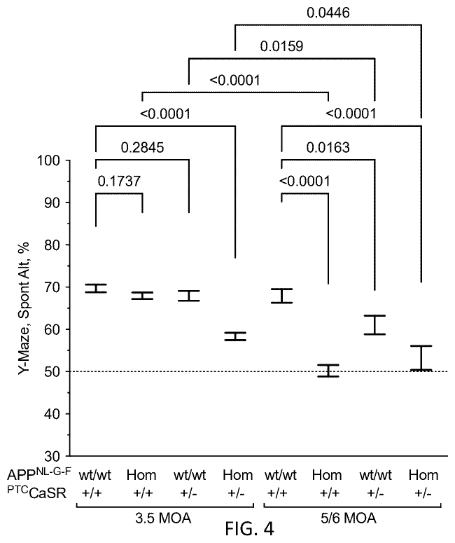

To assess the impact of HPT on early-onset AD (EOAD) due to genetic mutations in App gene, ^{PTC}CaSR^{Δflox/wt} mice were bred into the background of APP^{NL-G-F} mice, which carry humanized App gene with the 4 mutations identified from patients manifesting EOAD (Saito, T. et al. Nature neuroscience 17, 661-663 (2014); and Sasaguri, H. et al. EMBO J 36, 2473-2487 (2017). The homozygous APP^{NL-G-F} mice showed significant cognitive decline around 5-6 months of age (FIG. 4, blue triangles). Interestingly, HPT due to concurrent heterozygous deletion of the Casr gene in the parathyroid cell accelerates the cognitive declines as early as 3.5 MOA (FIG. 4, purple diamonds), demonstrating a synergistic action between HPT and Aβ access in promoting cognitive function declines.

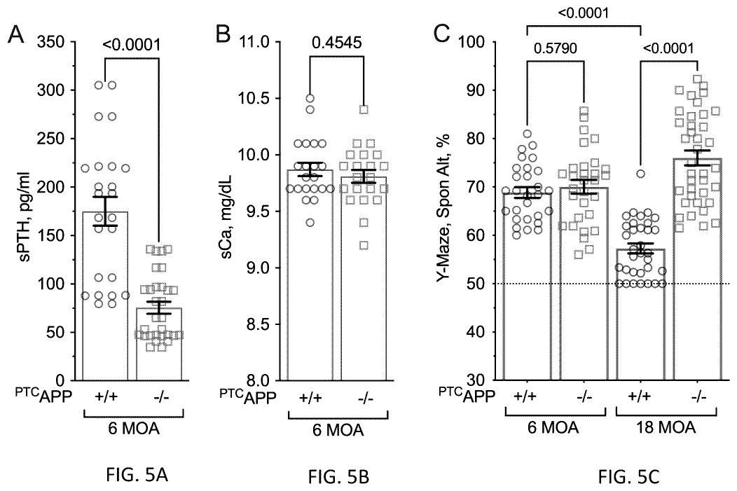

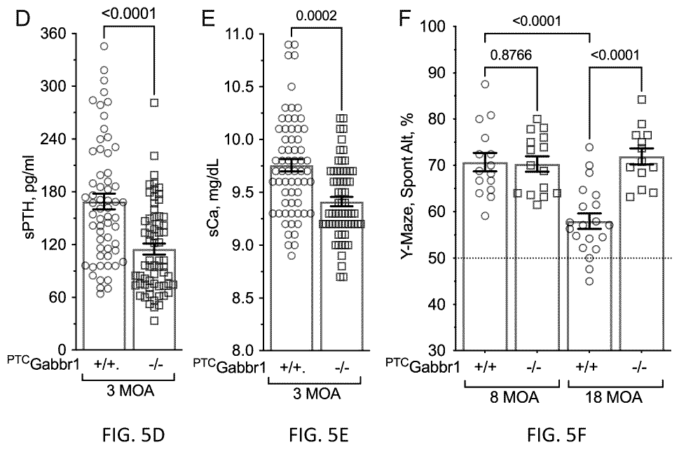

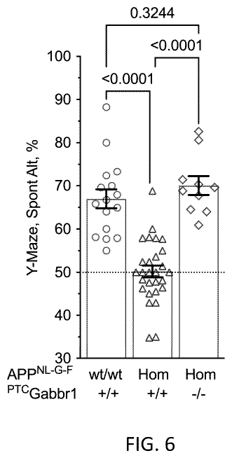

In contrast, aging-induced cognitive decline can be prevented in two mouse models of hypoparathyroidism, due to reduced tonic PTH secretion as the results of PTC-targeted ablation of amyloid precursor protein (APP) (^{PTC}APP^{Δflox/Δflox}) (FIGS. 5A-C) or type B γ-aminobutyric acid (GABA) receptor 1 (GABAB1R) (^{PTC}GABBR1^{Δflox/Δflox}) (Chang, W. et al. Nat Metab 2, 243-255, 2020) (FIGS. 5D-F). The ^{PTC}APP^{Δflox/Δflox} and ^{PTC}GABBR1^{Δflox/Δflox} mice showed reduced serum PTH levels before 3 months of age (FIGS. 5A,D) with hypocalcemia manifested in the ^{PTC}GABBR11^{Δflox/Δflox} mice (FIG. 5E). While their respective control littermates (+/+) showed clear cognitive declines at 18 MOA (FIGS. 5C,F), both ^{PTC}APP^{Δflox/Δflox} and ^{PTC}GABBR1^{Δflox/Δflox} mice retained normal cognitive functions at this age. Furthermore, crossing APP^{NL-G-F/NL-G-F} mice into ^{PTC}GABBR1^{Δflox/Δflox} background delay cognitive declines seen in the former mice at 6 MOA (FIG. 6).

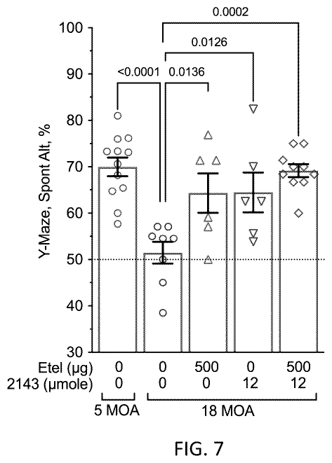

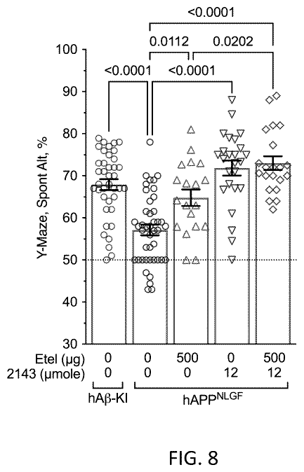

Based on the outcomes of the above genetically manipulated mice, it was tested whether pharmacological manipulations of serum PTH levels with blood-brain barrier (BBB)-impermeable calcimimetics (e.g., etelcalcetide) that activate CaSR homodimers in PTCs to suppress PTH secretion can prevent the cognitive declines in the aging and APP^{NL-G-F/NL-G-F} mice. Indeed, the cognitive declines seen in 18 MOA verse 5 MOA male C57/B6 mice (FIG. 7, blue vs gray circles) could be partially prevented by daily injections of etelcalcetide (blue circles vs. red triangles). Similarly, daily injections of etelcalcetide partially prevented early cognitive declines seen in the ^{APPNL-G-F} mice (FIG. 8, blue circles vs. red triangles).

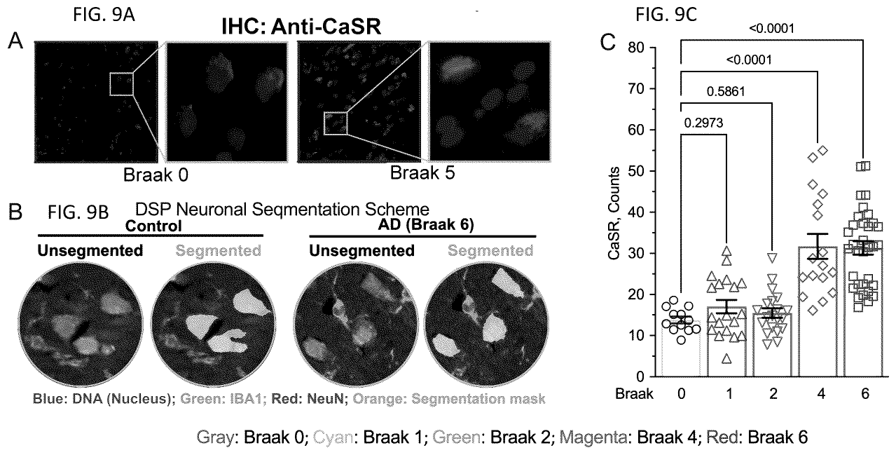

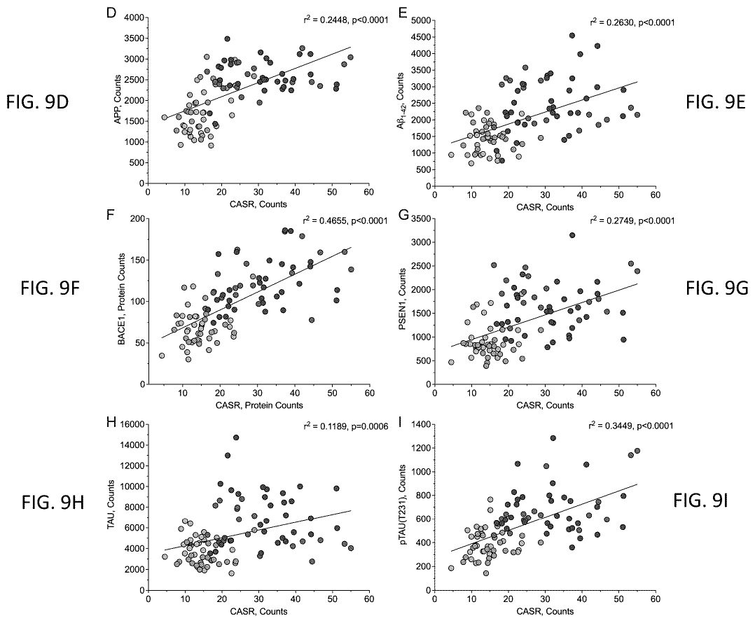

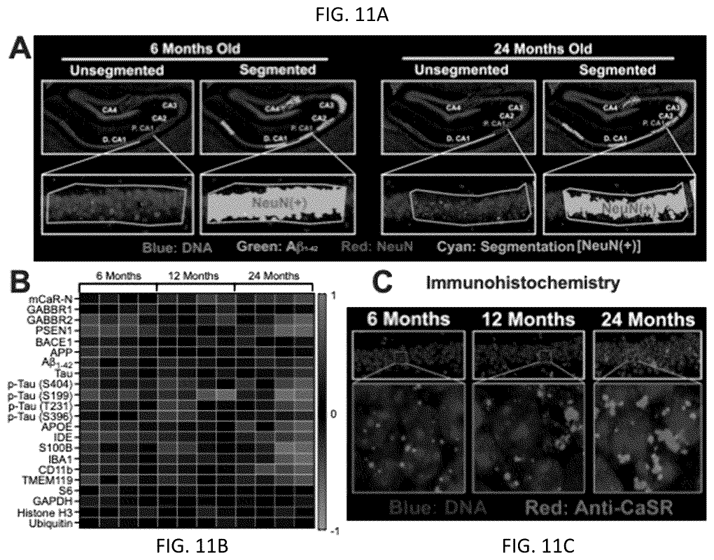

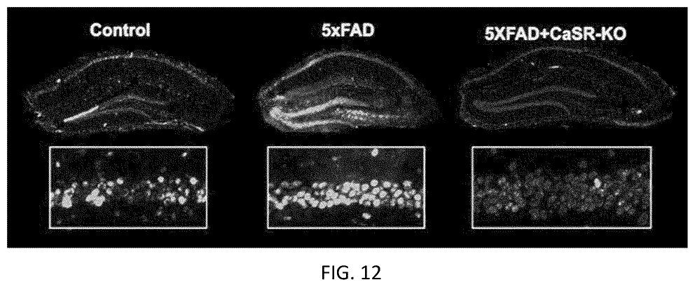

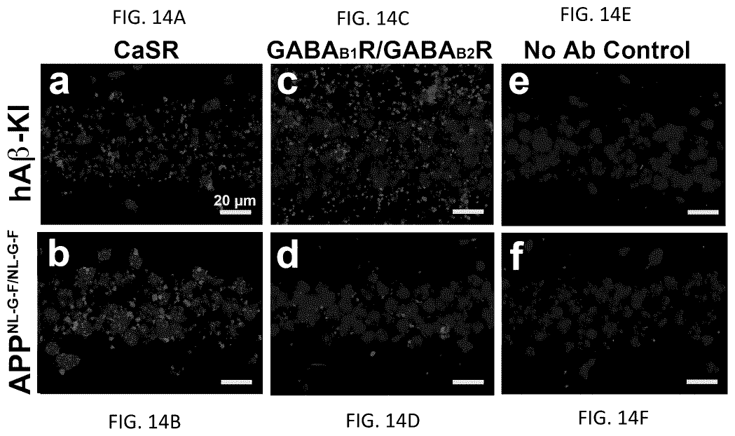

CaSR overexpression was previously shown in hippocampal neurons to cause neurodegeneration in multiple mouse models of brain injuries, by forming heterodimeric complexes with GABA_{B1}R and GABA_{B2}R to block Gi signaling of the latter receptors (Chang, W. et al. J Biol Chem 282, 25030-25040 (2007); and Chang, W. et al. Nat Metab 2, 243-255 (2020)). Likewise, the results disclosed herein show increased neuronal CaSR overexpression, which correlates with the appearance of AD hallmarks in the brains of AD patients (FIG. 9), aging mice (FIG. 11), and mouse models of EOAD (FIG. 12 and FIG. 14).

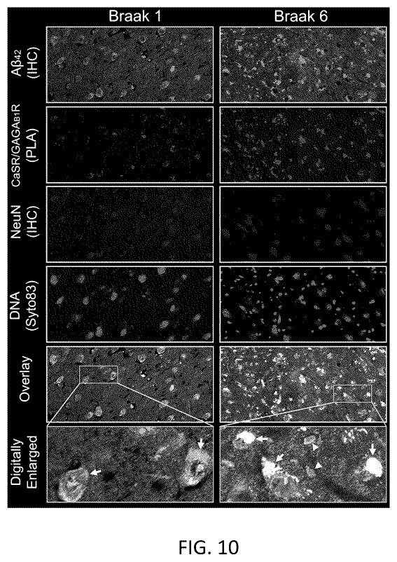

Next, it was examined whether Ab₄₂ closely co-localizes with CaSR/GABA_{B1}R heterodimers as a first step to establish their functional linkage. As disclosed herein, the data shows profound increases in the expression of Ab₄₂ (FIG. 10, green) and CaSR/GABA_{B1}R heterodimers (red), particularly in the NeuN(+) (blue) CA1 hippocampal neurons of Braak 6 vs. Braak 1 patients. As shown in the overlay images, Ab42 closely co-localizes with the CaSR/GABA_{B1}R dimers in the intracellular compartments of the neuron (shown in white color (due to merging of RGB colors) and indicated by while arrows in the digitally enlarged images). While some Aβ₄₂ signals (green) were observed in the NeuN(−) cells with dense Syto83 nuclear stain (magenta), minimal CaSR/GABA_{B1}R dimers were observed in those cells (white arrowheads in the digitally enlarged images), demonstrating neuron-centric actions of the CaSR/GABA_{B1}R heterodimers.

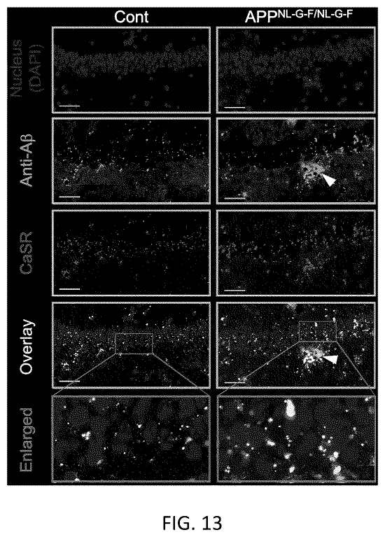

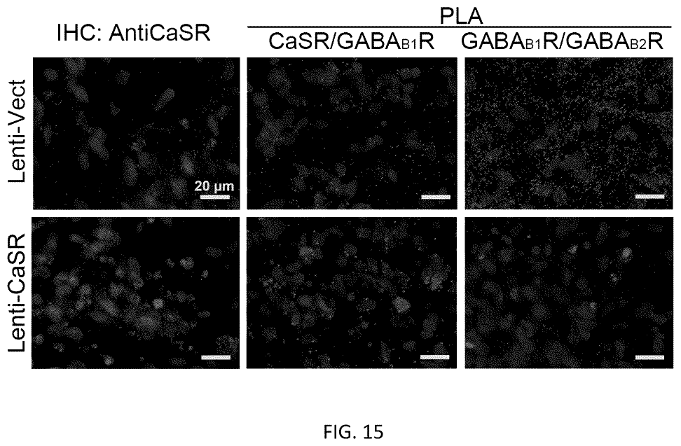

In the EOAD APP^{NL-G-F} mice, increased CaSR expression and its co-localization with Aβ₄₂ (FIG. 13) are associated with reduced heterodimerization of GABA_{B1}R and GABA_{B2}R (FIG. 14), which mediates important inhibitory neurotransmission to prevent excitotoxicity in normal physiology. The ability of CaSR overexpression to disrupt GABA_{B1}R/GABA_{B2}R signaling is further corroborated by the increased CaSR/GABA_{B1}R and reduced GABA_{B1}R/GABA_{B2}R heterodimerization in cultures of iPSC-derived neurons that overexpress CaSR thru infection with a lentiviral construct (Lenti-CaSR) (FIG. 15), supporting the ability of CaSR to disrupt normal GABA_{B}R signaling.

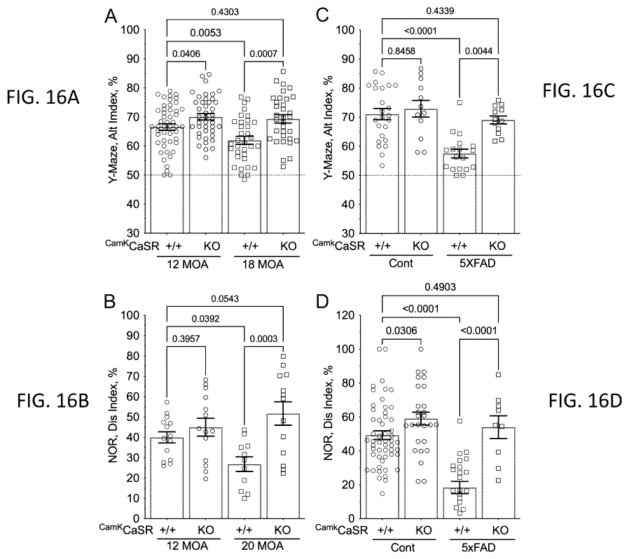

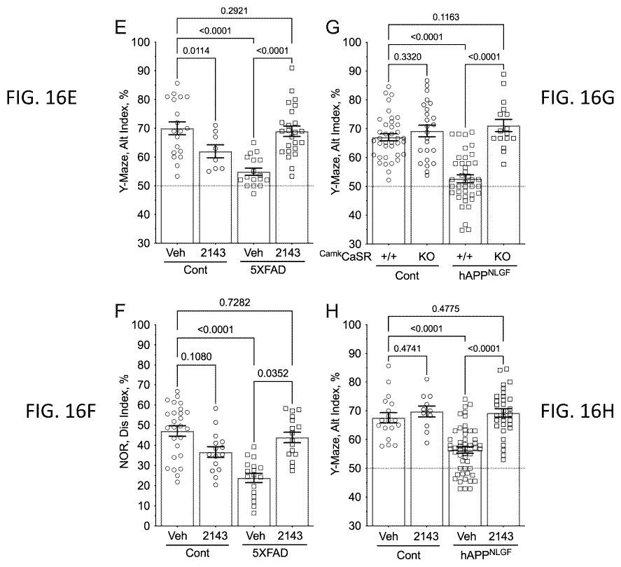

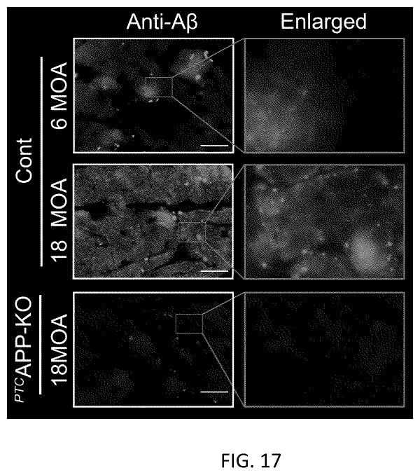

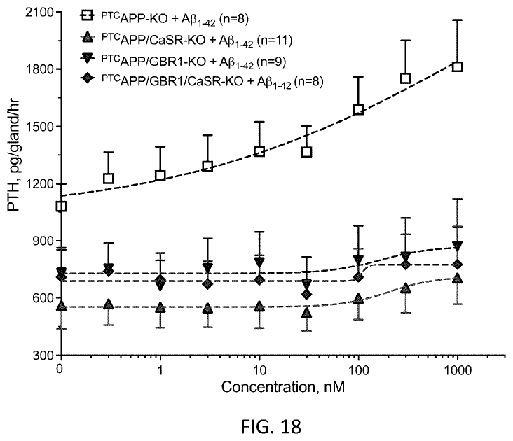

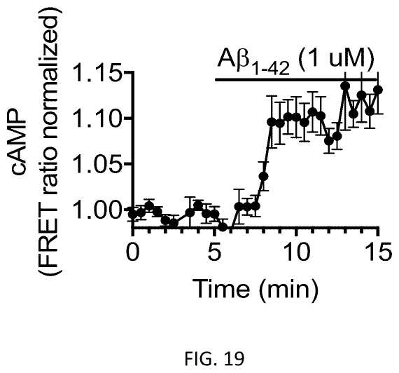

Suppressing CaSR activity by genetic ablation of neuronal CaSR or by daily injections of BBB-permeable calcilytics (negative allosteric modulators of CaSRs) alleviate or prevent cognitive declines in aging (FIGS. 16A-16B) and AD (FIGS. 16C-16F for 5×FAD mice and FIGS. 16G-16H for APP^{NLGF/NLGF}) mice, demonstrating a role of neuronal CaSR overexpression in AD development and progression. Given that HPT can cause chronic inflammation (Cheng, S. P., et al. Mediators Inflamm 2014, 709024 (2014); çrakolu, M. F. et al. ENT Updates 11, 115-119 (2021); and Almqvist, E. G., et al. Scandinavian journal of clinical and laboratory investigation 71, 139-144 (2011)) and that CaSR expression increases in inflammatory responses in pathological conditions (Jager, E. et al. Nat Commun 11, 4243 (2020); and Iamartino, L. & Brandi, M. L. Front Physiol 13, 1059369 (2022)), including in AD, through interleukin (IL)-1β (IL1β) and IL-6-induced NFkb cis-acting response of CASR gene (Canaff, L. & Hendy, G. N. J Biol Chem 280, 14177-14188 (2005); and Hendy, G. N. & Canaff, Semin Cell Dev Biol 49, 37-43 (2016)), HPT-induced inflammation could be a leading cause for CaSR upregulation as well as for Aβ buildup in AD (Kinney, J. W. et al. Alzheimers Dement (N Y) 4, 575-590 (2018); Ilievski, V. et al. PloS one 13, e0204941 (2018); and Yamamoto, M. et al. Am J Pathol 170, 680-692 (2007)). Furthermore, the results disclosed herein showed upregulation of Aβ₄₂ expression with age from 6 to 18 MOA in the PTGs of male App^{fl/fl} control, but not ^{PTC}App^{-/-}, mice (FIG. 17), indicating a role for the Aβ signaling cascade in promoting PTH hypersecretion. The studies disclosed herein showed investigating effects of Aβ in promoting tonic PTH secretion in PTCs (FIG. 18) and in stimulating cAMP production in iPSC-derived neurons co-expressing CASR and GABAB1R (FIG. 19) show that Aβ can serve as a co-agonist to activate CaSR/GABAB1R.

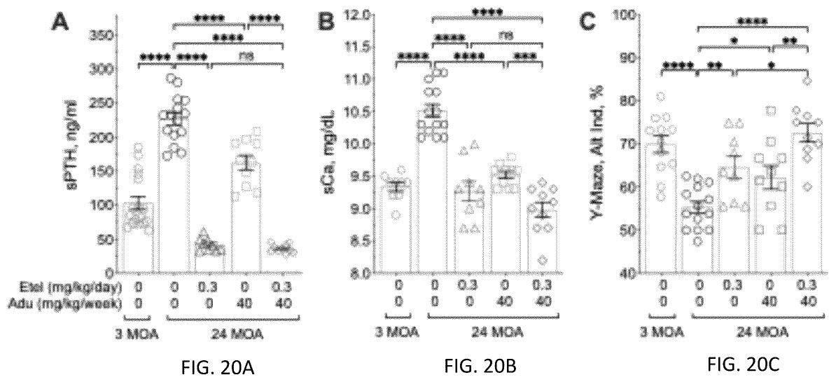

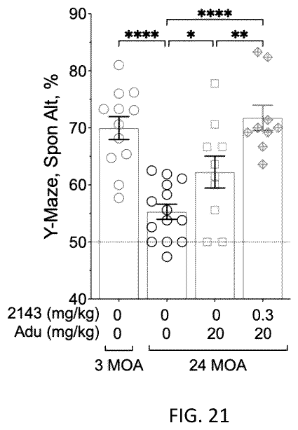

The studies disclosed herein tested whether reducing Aβ₄₂ levels using the recombinant Aβ-neutralizing antibody aducanumab (Adu) developed to slow cognitive decline in AD patients, could lessen aging-associated HPT. In vivo administration of aducanumab (40 mg/kg/week by twice weekly injections for 5 weeks) reduced the sPTH by 25% while daily injections of Etelcalcetide (0.3 mg/kg, once daily) reduced sPTH by >80% (FIG. 20A) but latter produced much more severe hypocalcemia (FIG. 20B). Co-treatment of Adu with Etel reduced sPTH and sCa to the levels comparable to the treatment of Etel alone (FIGS. 20A, 20B). However, combined Adu and Etel treatment produced more robust neuroprotection than each treatment alone (FIG. 20C), demonstrating synergistic effects of the Adu with Etel. The data disclosed herein show similar synergistic effects of Adu and BBB-permeable calcilkytics in restoring cognitive fucntions in aged mice (FIG. 21).

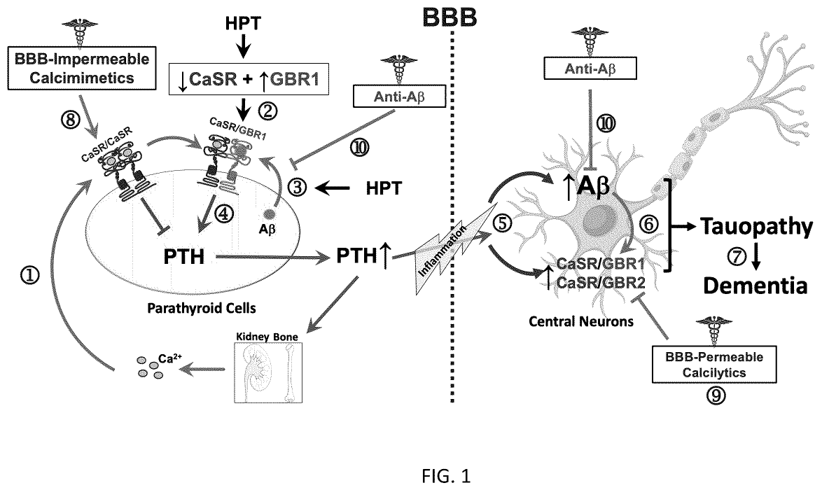

The data described herein support the working model and pharmaceutical regimens described in FIG. 1. Briefly, at the normal state (\textcircled{1}), CaSR homodimer is a dominant form that responds to increases in ambient [Ca²⁺] by suppressing PTH secretion through Gq signaling in PTCs to prevent excessive PTH-mediated hypercalcemic activities in kidney and bone. In this state, little CaSR is expressed in neurons, permitting Gi signaling of the dominant GABA_{B1}R/GABA_{B2}R heterodimers to balance excitable neurotransmission to prevent excitotoxicity. In aging or HPT states due to vitamin D deficiency (\textcircled{2}), reduced CaSR and increased GABA_{B1}R expression promote formation of CaSR/GABA_{B1}R heterodimers and expression of their co-ligands (Aβ) (\textcircled{3}) that together promote tonic PTH secretion, therefore leading to HPT (\textcircled{4}) that to produce systemic and central inflammatory responses and other undefined mechanisms (\textcircled{4}). These systemic changes upregulate neuronal CaSR expression (\textcircled{5}) to compete for dimerization with GABA_{B1}R and GABA_{B2}R to interfere with GABA_{B1}R/GABA_{B2}R-meidated Gi signaling as well as stimulate the ability of CaSR/GABA_{B1}R to suppress Gi signaling through mechanisms to be defined and the ability of CaSR/GABA_{B2}R to promote excitable Gq signaling. Inflammation may also increase neuronal synthesis of Aβ (\textcircled{5}) that further enhances activation of neuronal CaSR/GABA_{B1}R (\textcircled{6}) to promote tauopathy, excitotoxicity, and subsequent cognitive declines (\textcircled{7}). Based on this model, the following treatment regimens can be useful to prevent and/or reduce AD development or progression: (i) use of the BBB-impermeable calcimimetics (e.g., etelcalcetide) that can effectively suppress PTH secretion by activating CaSR homodimers in PTCs (\textcircled{8}) without impact on the neuronal CaSR to prevent HPT and its associated inflammation for early-stage AD patients; (ii) use of a combination of BBB-permeable calcilytics (e.g., NPS-2143) to directly suppress the activity of CaSR in the forms of CaSR/GABAB1R and CaSR/GABAB2R heterodimers in central neurons (\textcircled{9}) along with adequate dose to etelcalcetide to counter the action of calcilytics on CaSR in PTCs to prevent the development of unwanted HPT (\textcircled{8}+\textcircled{9}); (iii) uses of regimen (i) and/or (ii) along with anti-Aβ therapies (e.g., aduhelm) particularly for late stage AD patients (\textcircled{8}+\textcircled{10}, \textcircled{9}+\textcircled{10}, or \textcircled{8}+\textcircled{9}+\textcircled{10}).

To delineate the role of serum PTH in mediating inflammation, neuronal CaSR expression, Aβ metabolism, tauopathy, and cognition/memory function in AD (i) impact HPT (^{PTC}CaSR^{Δflox/wt} or ^{PTC}VDR^{Δflox/Δflox} mice), hypoparathyroidism (^{PTC}APP^{Δflox/Δflox} or PTC^{GABA}_{B1}R^{Δflox/Δflox} mice), and long-term injections of a long-acting PTH analog (LA-PTH) on these disease parameters in aging and APP^{NL-G-F/NL-G-F} mice will be carried out and (ii) PTH levels with pathologies in patients at different stages of AD will be correlated.

To delineate the molecular actions of CaSR overexpression to interfere with GABA_{B1}R/GABA_{B2}R-mediated Gi-signaling and to promote formation and excitable signaling of CaSR/GABA_{B1}R and CaSR/GABA_{B2}R and examine the ability of Aβ to bind and activate CaSR/GABA_{B1}R heterodimer (a) comparing the expression of CaSR, GABA_{B1}Rs, GABA_{B2}R, and their respective heterodimers in neurons in the brains of (i) aging and APP^{NL-G-F/NL-G-F} mice with or without neuronal CaSR KO and (ii) patients at different stages of AD vs controls will be carried out; (b) examining the impact of CaSR overexpression in the presence or absence of Aβ on the expression of CaSR, GABA_{B1}R, GABA_{B2}R, and their heterodimers, Gi-mediated cAMP production, and AD pathology in iPSC-derived neurons will be performed; (c) comparing the GABA_{B}R-dependent electrophysiological (Ephys) responses in brain slices and EEG/EMG recordings in vivo in aging or APP^{NL-G-F/NL-G-F} mice with or without neuronal CaSR KO will be performed; and (d) comparing activating and inactivating structures (by Cryo-EM) of CaSR/CaSR, CaSR/GABA_{B1}R and CaSR/GABA_{B2}R dimers in the presence or absence of Ca²⁺, GABA, and/or Aβ will be carried out.

Devising the pharmaceutical regimens targeting CaSR to delay onset and/or progression of AD will be performed by comparing the abilities of calcimimetics (BBB-impermeable etelcalcetide), calcilytics (BBB-permeable NPS2143), anti-Aβ therapy (Aduhelm), or their different combinations to alleviate pathological (Aβ plaque, phosphorylated tau, and microglia activation), ephys (in vivo EEG/EMG recordings), and neurobehavioral phenotypes of AD in aging and APP^{NL-G-F/NL-G-F} mice.

### Example 2

B-Amyloid Mediates PTH Hypersecretion in Hyperparathyroidism Associated with Vitamin D Deficiency. Primary hyperparathyroidism (PHPT) is a common endocrinopathy characterized by elevated parathyroid hormone (PTH) secretion. Low serum 25-hydroxyvitamin D (25OHD) levels are more prevalent in PHPT patients than in the general population, however, the mechanistic basis for this association is unclear. Previous studies (Nat Metab 2:243) demonstrated that increased heterodimerization and co-activation (by GABA and Ca²⁺, respectively) of the type B γ-aminobutyric acid receptor 1 (GABA_{B1}R) and the extracellular Ca²⁺-sensing receptor (CaSR) promote tonic PTH secretion from the parathyroid glands (PTGs) isolated from PHPT patients and HPT mice.

In searching for additional ligands of the GABA_{B1}R/CaSR heterodimer, upregulation of a putative GABA_{B1}R ligand, the amyloid precursor protein (APP), and one of its derivatives, b-amyloid (Ab₁₋₄₂) in the parathyroid adenoma of PHPT patients verse age-matched normal PTGs (p<0.005) were found by in situ proteomic profiling. Those tumors also showed increased levels of the proteolytic enzymes, β-secretase and γ-secretase, that make Aβ₁₋₄₂ and enhanced phosphorylation of the microtubule-associated protein TAU, a downstream effector of Aβ₁₋₄₂-induced signaling in the degenerative neurons of dementia patients.

Adding exogenous Aβ₁₋₄₂ (0.3 to 1000 nM) in cultures concentration-dependently stimulated tonic PTH secretion by up to 1.6-fold in murine (p<0.001 vs control) and 1.9-fold in human PTGs (p=0.03 vs control) without shifting the Ca²⁺-set point. This stimulatory effect was absent in murine PTGs lacking either CaSR or GABA_{B1}R. Furthermore, parathyroid cell (PTC)-specific knockout (KO) of the App (^{PTC}App^{Dflox/Dflox}) gene to remove Aβ₁₋₄₂ or the Gabbr1 (^{PTC}Gabbr1^{Dflox/Dflox}) gene to disrupt GABA_{B1}R/CaSR heterodimer similarly reduced tonic PTH secretion in PTG cultures and produced hypoparathyroidism in vivo, demonstrating a role for Aβ₁₋₄₂ as a ligand in stimulating PTH secretion via GABA_{B1}R/CaSR heterodimer. The proteomic profiles revealed a significant inverse correlation between the pre-operative 25OHD levels of PHPT patients and increased Tau phosphorylation in their PTG tumors (r²=0.225, p<0.0001), demonstrating a role for Tau signaling in stimulating PTH secretion in 25OHD deficiency.

In support of this finding, inhibition of Tau phosphorylation by a staurosporine analogue (K252a) blocked the ability of Aβ₁₋₄₂ to stimulate PTH secretion in vitro, and the increased tonic PTH hypersecretion seen in mice with PTC-targeted Vdr gene KO (^{PTC}Vdr^{Dflox/Dflox}), mimicking vitamin D deficiency, was reversed by a concurrent ablation of App (^{PTC}Vdr^{Dflox/Dflox}; App^{Dflox/Dflox}) or the TAU-encoding Mapt (^{PTC}Vdr^{Dflox/Dflox}; Mapt−/−) gene. Collectively, the results described herein demonstrate roles of Aβ₁₋₄₂/p-Tau signaling in sustaining tonic PTH secretion in physiological states and in promoting PTH hypersecretion due to 25OHD deficiency.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1. A method of treating Alzheimer's disease or dementia in a subject, the method comprising administering to the subject a therapeutically effective amount of a blood-brain barrier (BBB)-impermeable calcimimetic or a blood-brain barrier-permeable calcilytic, thereby treating or preventing Alzheimer's disease or dementia in the subject.

2. A method of increasing calcium-sensing receptor (CaSR) homodimer formation, increasing expression or activity of a calcium-sensing receptor (CaSR) homodimer or blocking CaSR/GABA-B1 receptor heterodimer formation in both peripheral tissues and the central nervous system (CNS) in a subject, the method comprising administering to the subject a therapeutically effective amount of a blood-brain barrier (BBB)-impermeable calcimimetic and a calcilytic, thereby increasing calcium-sensing receptor (CaSR) homodimer formation, increasing expression or activity of CaSR homodimer or blocking CaSR/GABA-B1 receptor heterodimer formation in peripheral tissues and the CNS, respectively in the subject.

3. A method of reducing or blocking CaSR/GABA-B1 receptor heterodimer activity in a subject, the method comprising administering to the subject a therapeutically effective amount of a blood-brain barrier (BBB)-impermeable calcimimetic and a calcilytic, thereby reducing or blocking CaSR/GABA-B1 receptor heterodimer activity in the subject.

4. The method of claim 2, wherein calcium-sensing receptor (CaSR) homodimer formation, expression or activity of the CaSR homodimer is increased and CaSR/GABA-B1 receptor heterodimer formation is blocked in both peripheral tissues and in the central nervous system.

5. The method of claim 3, wherein the CaSR/GABA-B1 receptor heterodimer activity is reduced or blocked in both peripheral tissues and in the central nervous system.

6. The method of claim 2, wherein the therapeutically effective amount of a blood-brain barrier (BBB)-impermeable calcimimetic increases CaSR homodimer formation, increases expression or activity of the CaSR homodimer in peripheral tissues in the subject.

7. (canceled)

8. The method of claim 2, wherein the therapeutically effective amount of the blood-brain barrier (BBB)-impermeable calcilytic reduces CaSR/GABA-B1 receptor heterodimer formation, reduces expression or activity of the CaSR/GABA-B1 receptor heterodimer in the CNS in the subject.

9. The method of claim 2, wherein the increasing of CaSR homodimer formation, the increasing of the expression or activity of the CaSR homodimer occurs in peripheral tissues of the subject concurrently with the blocking of the CaSR/GABA-B1 receptor heterodimer formation in the CNS in the subject.

10.-12. (Canceled)

13. The method of claim 2, further comprising administering to the subject an anti-amyloid-beta (anti-Aβ) therapy.

14.-23. (canceled)

24. The method of claim 1, wherein the method reduces beta-amyloid plaque formation, phosphorylated tau, microglia activation or a combination thereof in the subject.

25. The method of claim 13, wherein the administration of the anti-amyloid-beta therapy reduces circulating and tissue beta-amyloid levels in the subject.

26. The method of claim 25, wherein the beta-amyloid level is reduced in neurons, in parathyroid cells, or both.

27. The method of claim 1, further comprising administering a therapeutically effective amount of a beta-amyloid synthesis inhibitor to the subject.

28. The method of claim 13, wherein the anti-Aβ therapy is lecanemab (Leqembi®), aducanumab (Aduhelm®), or donanemab.

29. (canceled)

30. (canceled)

31. The method of claim 1, further comprising administering a therapeutically effective amount of aducanumab-avwa to the subject.

32. (canceled)

33. The method of claim 1, wherein the blood-brain barrier (BBB)-impermeable calcimimetic is etelcalcetide, NPS2143, ATF-936, AXT-914, CLTX-305, or a combination thereof.

34.-37. (canceled)

38. The method of claim 1, wherein the subject has or is at risk for having Alzheimer's disease, dementia, stroke or a trauma-induced neuronal injury.

39. The method of claim 1, wherein said treating reduces or ameliorates one or more symptoms of Alzheimer's disease or dementia in the subject.

40. The method of claim 39, wherein the one or more symptoms of Alzheimer's disease or dementia is loss of cognition or memory and neurodegeneration.
